SEQUENCE LISTING (28 sequences)

SEQ ID NO: 1 - DNA, 15 bp, Artificial Sequence
  synthetic construct
ccagtgggcaggcgg

SEQ ID NO: 2 - DNA, 21 bp, Artificial Sequence
  synthetic construct
aggaactccttctccagctct

SEQ ID NO: 3 - DNA, 20 bp, Artificial Sequence
  synthetic construct
cgtgtcccagacgttctcag

SEQ ID NO: 4 - DNA, 20 bp, Artificial Sequence
  synthetic construct
ccttgtggggagagcttcag

SEQ ID NO: 5 - DNA, 21 bp, Artificial Sequence
  synthetic construct
gctctggagacttctgaacga

SEQ ID NO: 6 - DNA, 20 bp, Artificial Sequence
  synthetic construct
ccgttcttcaccgacttcct

SEQ ID NO: 7 - DNA, 19 bp, Artificial Sequence
  synthetic construct
gttcaccagccggactgaa

SEQ ID NO: 8 - DNA, 20 bp, Artificial Sequence
  synthetic construct
gcaggtcagtaacctcggac

SEQ ID NO: 9 - DNA, 21 bp, Artificial Sequence
  synthetic construct
tcaggacaaagcagtgctcaa

SEQ ID NO: 10 - DNA, 21 bp, Artificial Sequence
  synthetic construct
tgtccatcaagtgaaccccag

SEQ ID NO: 11 - DNA, 20 bp, Artificial Sequence
  synthetic construct
gcacccctatgaagacccag

SEQ ID NO: 12 - DNA, 22 bp, Artificial Sequence
  synthetic construct
ggactgtctggttgaattgtcg

SEQ ID NO: 13 - DNA, 18 bp, Artificial Sequence
  synthetic construct
gggagcggtgaagatgga

SEQ ID NO: 14 - DNA, 22 bp, Artificial Sequence
  synthetic construct
tcatgttgctcacggaggagta

SEQ ID NO: 15 - DNA, 19 bp, Artificial Sequence
  synthetic construct
agaagcgcgtgccttcatc

SEQ ID NO: 16 - DNA, 20 bp, Artificial Sequence
  synthetic construct
tttctgcgccataaggtggt

SEQ ID NO: 17 - DNA, 20 bp, Artificial Sequence
  synthetic construct
gtttcaccaagtgccggaag

SEQ ID NO: 18 - DNA, 20 bp, Artificial Sequence
  synthetic construct
cccattcctgggatgtcacc

SEQ ID NO: 19 - DNA, 20 bp, Artificial Sequence
  synthetic construct
gaacctgctccctcccagtc

SEQ ID NO: 20 - DNA, 19 bp, Artificial Sequence
  synthetic construct
gtgcatagtggcacctgct

SEQ ID NO: 21 - DNA, 20 bp, Artificial Sequence
  synthetic construct
gaacgcatccgattcatctt

SEQ ID NO: 22 - DNA, 20 bp, Artificial Sequence
  synthetic construct
cttgatccacctggctgttt

SEQ ID NO: 23 - DNA, 20 bp, Artificial Sequence
  synthetic construct
actgcaagatgatgcaggtg

SEQ ID NO: 24 - DNA, 20 bp, Artificial Sequence
  synthetic construct
atgaggctgtggaagtctgg

SEQ ID NO: 25 - DNA, 20 bp, Artificial Sequence
  synthetic construct
tgaccttctgcctcttacca

SEQ ID NO: 26 - DNA, 21 bp, Artificial Sequence
  synthetic construct
cactatcactggcactgttgc

SEQ ID NO: 27 - DNA, 21 bp, Artificial Sequence
  synthetic construct
ggctaaacaggtactgctggg

SEQ ID NO: 28 - DNA, 23 bp, Artificial Sequence
  synthetic construct
aggaaagccaggtacttcaactt

(19) United States
(12) Patent Application Publication
(10) Pub. No.: US 2024/0084262 A1
(43) Pub. Date: Mar. 14, 2024

(54) METHODS FOR DIFFERENTIATION OF PANCREATIC EXOCRINE CELLS FROM HUMAN INDUCED PLURIPOTENT STEM CELLS

(71) Applicant: CEDARS-SINAI MEDICAL CENTER, Los Angeles, CA (US)

(72) Inventors: Dhruv Sareen, Porter Ranch, CA (US); Stephen Pandol, Los Angeles, CA (US); Syairah Hanan Shaharuddin, Sun Valley, CA (US); Victoria Wang, San Diego, CA (US); Michael Edison P. Ramos, Perris, CA (US); Aurelia Lugea, Los Angeles, CA (US); Richard T. Waldron, Los Angeles, CA (US)

(73) Assignee: CEDARS-SINAI MEDICAL CENTER, Los Angeles, CA (US)

(21) Appl. No.: 18/268,822

(22) PCT Filed: Oct. 29, 2021
(86) PCT No.: PCT/US2021/057403

§ 371 Date: Jun. 21, 2023

Related U.S. Application Data
(60) Provisional application No. 63/130,214, filed on Dec. 23, 2020.

(51) Int. Cl. C12N 5/071; C12M 1/00; C12M 1/12; C12M 3/06; C12N 5/00; G01N 33/50

(52) U.S. Cl. CPC C12N 5/0677; C12M 23/16; C12M 23/34; C12M 25/02; C12M 25/14; C12N 5/0031; G01N 33/507; C12N 2500/32; C12N 2500/38; C12N 2500/90; C12N 2501/11; C12N 2501/115; C12N 2501/119; C12N 2501/155; C12N 2501/16; C12N 2501/385; C12N 2501/41; C12N 2501/415; C12N 2506/45

(57) ABSTRACT

The present invention provides for methods of differentiating induced pluripotent stem cells into pancreatic progenitor cells, pancreatic ductal cells, pancreatic endocrine cells, pancreatic acinar cells, and pancreatic organoids. Cells created by these methods are also provided. Further provided are disease models and methods of drug screening.

### CROSS-REFERENCE TO RELATED APPLICATIONS

This application includes a claim of priority under 35 U.S.C. § 119(e) to U.S. provisional patent application No. 63/130,214 filed Dec. 23, 2020, the entirety of which is hereby incorporated by reference.

### FIELD OF INVENTION

This invention relates to generating pancreatic cells from human induced pluripotent stem cells and using these cells for disease models and development of therapeutics.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

While several protocols exist for the differentiation of pancreatic beta cells from hiPSCs and for the differentiation of pancreatic exocrine cells from mouse embryonic cells or human pluripotent cells, none exist for the reliable and robust generation of pancreatic exocrine cells from hiPSCs in multiple formats.

Further, no treatments exist for pancreatitis, and the molecular mechanisms of both ductal and acinar cell carcinomas are not completely understood due to a lack of viable models.

In vitro differentiation has been performed with mouse embryonic cells. However, limited protocols exist with efficient pancreatic exocrine cell differentiation for hiPSCs in the past, and none with hiPSCs in both adherent and organoid cultures robustly. As for in vitro modeling using primary cells obtained from humans, acinar cells do not maintain functionality in culture for more than a couple days, and ductal cells are very difficult to isolate.

Pancreatitis and pancreatic cancer modeling exist using animal models, however it is difficult to create human tissue models with uniform pancreatic damage and studying of the tissue necessitates animal sacrifice and animals do not accurately reflect the human conditions that exist in pancreatic exocrine diseases.

As such, there remains a need in the art for reliable and robust generation of pancreatic exocrine cells from hiPSCs in multiple formats, as well as the ability to study and develop treatments for pancreatitis, and ductal and acinar cell carcinoma.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of differentiating induced pluripotent stem cells (iPSCs) into iPSC-derived pancreatic progenitor cells, comprising: seeding induced pluripotent stem cells (iPSCs) on a solid medium coated with solubilized basement membrane preparation in the presence of serum-free, stabilized cell culture medium, and optionally a ROCK inhibitor; culturing the cells in a first culture medium comprising a first base medium, Activin A, CHIR99021, and a ROCK Inhibitor; culturing the cells in a second culture medium comprising the first base medium and Activin A, and bFGF (FGF-2); culturing the cells in a third culture medium comprising the first base medium and FGF10, NOGGIN, and CHIRR99021; culturing the cells in a fourth culture medium comprising a second base medium and FGF10, NOGGIN, All-trans Retinoic Acid, and SANT1, wherein iPSC-derived pancreatic progenitor cells are produced.

In various embodiments, the Activin A, CHIR99021, and the ROCK Inhibitor in the first culture medium can be at a concentration of about 100 ng/ml Activin A, about 2 uM CHIR99021 and about 10 uM of the ROCK inhibitor. In various embodiments, the Activin A, and bFGF (FGF-2) in the second culture medium can be at a concentration of about 100 ng/ml Activin A and about 5 ng/ml bFGF (FGF-2). In various embodiments, the FGF10, NOGGIN, and CHIRR99021 in the third culture medium can be at a concentration of about 50 ng/ml FGF10, about 50 ng/ml NOGGIN and about 0.25 uM CHIRR99021. In various embodiments, the FGF10, NOGGIN, All-trans Retinoic Acid, and SANT1 in the fourth culture medium can be at a concentration of about 50 ng/ml FGF10, about 50 ng/ml NOGGIN, about 2 uM All-trans Retinoic Acid, and about 0.25 uM SANT1.

In various embodiments, the cells can be cultured in the first culture medium for about 1 day. In various embodiments, the cells can be cultured in the second culture medium for about 1 to 2 days. In various embodiments, the cells can be cultured in the third culture medium for about 1 to 2 days. In various embodiments, the cells can be cultured in the fourth culture medium for about 1 to 2 days.

Various embodiments of the present invention provide for a method of differentiating induced pluripotent stem cells (iPSCs) into induced pancreatic (iPan) ductal cells, comprising: performing the method of differentiating induced pluripotent stem cells (iPSCs) into iPSC-derived pancreatic progenitor cells, or providing iPSC-derived pancreatic progenitor cells; seeding the iPSC-derived pancreatic progenitor cells on a solid medium coated with solubilized basement membrane preparation in the presence of a fifth culture medium comprising a second base medium and a ROCK inhibitor; culturing the cells in a sixth culture medium comprising the second base medium and FGF10, EGF, and sDLL-1, wherein iPan ductal cells are produced.

In various embodiments, the method can further comprise dissociating the iPSC-derived pancreatic progenitor cells before seeding the iPSC-derived pancreatic progenitor cells on the solid medium.

In various embodiments, the ROCK inhibitor in the fifth culture medium can be at a concentration of about 10 uM. In various embodiments, FGF10, EGF, and sDLL-1 in the sixth culture medium can be at a concentration of about 25 ng/ml FGF10, about 50 ng/ml EGF, and about 50 ng/ml sDLL-1.

In various embodiments, the cells can be cultured in the fifth culture medium for about 1 day. In various embodiments, the cells can be cultured in the sixth culture medium for about 16 days.

Various embodiments of the present invention provide for a method of differentiating induced pluripotent stem cells (iPSCs) into induced pancreatic endocrine cells, comprising: performing the method of differentiating induced pluripotent stem cells (iPSCs) into iPSC-derived pancreatic progenitor cells; continuing to culturing the cells in the fourth culture medium comprising a second base medium and FGF10, NOGGIN, All-trans Retinoic Acid, and SANT1, for at least 2 additional days; seeding the iPSC-derived pancreatic progenitor cells on a solid medium coated with solubilized basement membrane preparation in the presence of a fifth culture medium comprising a second base medium and a ROCK inhibitor; culturing the cells in a sixth culture medium comprising the second base medium and noggin, EGF, and nicotinamide, wherein pancreatic endocrine cells are produced.

Various embodiments of the present invention provide for a method of differentiating induced pluripotent stem cells (iPSCs) into induced pancreatic endocrine cells, comprising: providing iPSC-derived pancreatic progenitor cells that have been cultured in the fourth culture medium comprising a second base medium and FGF10, NOGGIN, All-trans Retinoic Acid, and SANT1, for at least 2 additional days; seeding the iPSC-derived pancreatic progenitor cells on a solid medium coated with solubilized basement membrane preparation in the presence of a fifth culture medium comprising a second base medium and a ROCK inhibitor; culturing the cells in a sixth culture medium comprising the second base medium and noggin, EGF, and nicotinamide, wherein pancreatic endocrine cells are produced.

Various embodiments of the present invention provide for a method of producing pancreatic organoids comprising pancreatic ductal cells, comprising: performing a method of differentiating induced pluripotent stem cells (iPSCs) into iPSC-derived pancreatic progenitor cells and dissociated the iPSC-derived pancreatic progenitor cells, or providing dissociated iPSC-derived pancreatic progenitor cells; seeding the cells into a solubilized basement membrane preparation and plating the seeded cells onto a solid medium; and culturing the cells in a sixth culture medium comprising the second base medium and FGF10, EGF, and sDLL-1.

In various embodiments, the iPSC-derived pancreatic progenitor cells are resuspended in the solubilized basement membrane preparation at a density of about 2 million cells/ML.

In various embodiments, plating the seeded cells onto a solid medium can comprise plating about 10 μL of cell suspension onto each solid medium.

In various embodiments, the cells can be cultured in the sixth culture medium for at least 2 weeks and given the sixth culture medium about every 2-3 days.

In various embodiments, the solid medium is a U-bottom multi-well plate.

Various embodiments of the present invention provide for a method of differentiating induced pluripotent stem cells (iPSCs) into iPSC derived pancreatic acinar cells, comprising: performing a method of differentiating induced pluripotent stem cells (iPSCs) into iPSC-derived pancreatic progenitor cells, or providing iPSC-derived pancreatic progenitor cells; seeding the iPSC-derived pancreatic progenitor cells into a solubilized basement membrane preparation and plating the seeded cells onto a solid medium; culturing the cells in a seventh culture medium comprising a second base medium and XXI, FGF10, Noggin, Nicotinamide, and Murine Wnt3a, wherein iPSC derived pancreatic acinar cells are produced.

In various embodiments, the XXI, FGF10, Noggin, Nicotinamide, and Murine Wnt3a in the seventh culture medium can be at a concentration of about 20 ng/mL FGF10, about 25 ng/mL Wnt3a, about 1 uM XXI, about 50 ng/mL Noggin, about 10 mM Nicotinamide.

Various embodiments of the present invention provide for a method of producing organoids comprising pancreatic acinar cells, comprising: performing a method of differentiating induced pluripotent stem cells (iPSCs) into iPSC derived pancreatic acinar cells, or providing iPSC-derived pancreatic progenitor cells and performing a method of differentiating induced pluripotent stem cells (iPSCs) into iPSC derived pancreatic acinar cells, and dissociated the iPSC-derived pancreatic acinar cells, or providing dissociated iPSC-derived pancreatic acinar cells; seeding the iPSC-derived pancreatic acinar cells into a solubilized basement membrane preparation and plating the seeded cells onto a solid medium; and culturing the cells in an eighth culture medium comprising a second base medium and FGF10, Noggin, Nicotinamide, and Murine Wnt3a.

In various embodiments, seeding the iPSC-derived pancreatic acinar cells can comprise seeding at a concentration of about 1-3×10\hat{ }6 cells per about 1040 μL of the solubilized basement membrane preparation.

In various embodiments, plating the seeded cells onto a solid medium can comprise plating in about 15 μL bubbles and allowing the plated cells to sit for about 15 minutes prior to culturing the cells in the eighth culture medium.

In various embodiments, the cells can be cultured in the eighth culture medium for about 44 days, and given the eighth culture medium about every other day.

In various embodiments, the solid medium can be a round-bottom multi-well plate.

In various embodiments, the method can further comprise inducing PTF1A expression in the iPSC-derived pancreatic progenitor cells. In various embodiments, inducing PTF1A expression in the iPSC-derived pancreatic progenitor cells can comprise transducing the iPSCs, pancreatic progenitor cells, or both with a lentivirus vector comprising a nucleic acid encoding PTF1A.

In various embodiments, the solubilized basement membrane preparation used in these methods can be extracted from the Engelbreth-Holm-Swarm (EHS) mouse sarcoma.

In various embodiments, the solubilized basement membrane preparation used in these methods can be MATRIGEL matrix.

In various embodiments, the ROCK inhibitor used in these methods can be Y27632 ROCK Inhibitor.

In various embodiments, the serum-free, stabilized cell culture medium used in these methods can be mTeSR™ plus medium.

In various embodiments, the first base medium used in these methods can comprise an agent from the group consisting of MCDB 131, Glutamax, PSA, Vitamin C, BSA, sodium bicarbonate, antibiotic, and combinations thereof.

In various embodiments, the first base medium used in these methods can comprise MCDB 131, Glutamax, Vitamin C, BSA, sodium bicarbonate, and antibiotic.

In various embodiments, the Glutamax, Vitamin C, BSA, sodium bicarbonate, and antibiotic used in these methods can be at a concentration of about 1×Glutamax, about 1×Antibiotic, about 0.5% Bovine Serum Albumin, about 1.5 mg/ml Sodium Bicarbonate and about 250 uM Vitamin C.

In various embodiments, the second base medium used in these methods can comprise an agent from the group consisting of DMEM, antibiotic, B27 without vitamin A, vitamin C and combination thereof.

In various embodiments, the second base medium used in these methods can comprise DMEM, antibiotic, B27 without vitamin A, and vitamin C.

In various embodiments, the antibiotic, B27 without vitamin A, and uM vitamin C used in these methods can be at a concentration of about 1×antibiotic, about 1×B27 without vitamin A, and about 250 uM vitamin C.

In various embodiments, the antibiotic used in these methods can be a Pen-Strep Antibiotic.

Various embodiments of the present invention provide for an induced pluripotent stem cell (iPSC) derived pancreatic progenitor cell made by a method of the present invention.

Various embodiments of the present invention provide for an induced pluripotent stem cell (iPSC) derived iPan ductal cell made by a method of the present invention.

Various embodiments of the present invention provide for an induced pluripotent stem cell (iPSC) derived organoid comprising pancreatic ductal cells made by a method of the present invention.

Various embodiments of the present invention provide for an induced pluripotent stem cell (iPSC) derived pancreatic acinar cell made by a method of the present invention.

Various embodiments of the present invention provide for an induced pluripotent stem cell (iPSC) derived organoid comprising pancreatic acinar cells made by a method of the present invention.

Various embodiments of the present invention provide for a model, comprising a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; and a fluidic device, or a cell culture plate, or a multi-well culture plate, wherein the test agent and the population of cells are in contact in a fluidic device the cell culture plate, or the multi-well culture plate.

In various embodiments, the fluidic device, the cell culture plate, or the multi-well culture plate can be a Transwell system. In various embodiments, the fluidic device can be a microfluidic device. In various embodiments, the microfluidic device can be an organ chip.

Various embodiments of the present invention provide for a device, comprising: a membrane comprising a top surface and a bottom surface; a first channel in fluidic communication with the top surface of the membrane; a second channel in fluidic communication with the bottom surface of the membrane, wherein the first and second channels each comprises a surface that is parallel to the membrane; a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells in the first channel or the second channel; and optionally, a second population of cells in the first channel or the second channel, wherein the first population of cells and the second population of cells are in different channels, and wherein the first population of cells and the second population of cells are not the same type of cells.

In various embodiments, the second population of cells can be selected from the group consisting of endocrine cells, stellate cells endothelial cells, pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells.

In various embodiments, the device can further comprise at least one inlet port adapted for fluid entering the at least one inlet port; and at least one outlet port adapted for fluid exiting the at least one outlet port.

Various embodiments of the invention provide for a device, comprising: a top chamber; a bottom chamber; a membrane between the top chamber and the bottom chamber; a first channel fluidically coupled to the top chamber; a second channel fluidically coupled to the bottom chamber; a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells in the first channel or the second channel; and optionally, a second population of cells in the first channel or the second channel, wherein the first population of cells and second population of cells are in different channels, and wherein the first population of cells and the second population of cells are not the same type of cells.

In various embodiments, the second population of cells can be selected from the group consisting of endocrine cells, stellate cells endothelial cells, pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells.

In various embodiments, the first and second channels can comprise polydimethylciloxane. In various embodiments, the first channel and the second channel can be microfluidic channels.

Various embodiments of the present invention provide for an organ chip device, comprising: a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells; optionally, a second population of cells; and a membrane separating the first population of cells and the second population of cells, wherein the first population of cells and the second population of cells are not the same type of cells.

In various embodiments, the membrane can comprise polydimethylciloxane.

In various embodiments, the device can further comprise one or more gels and the population of cells having been seeded on top of or into the one or more gels.

In various embodiments, the first population of cells, or the second population of cells or both can be patient specific.

In various embodiments, the first population of cells, or the second population of cells or both can express a fluorescent reporter.

Various embodiments of the present invention provide for a method of assessing a test agent, comprising: contacting the test agent to a device of the present invention, wherein the device comprises a population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells, and optionally, a second population of cells; measuring a parameter; and assessing the test agent based on the measured parameter.

In various embodiments, measuring the parameter can comprise measuring a phenotype of interest, expression level of a gene of interest, or expression level of a protein of interest, or combinations thereof.

In various embodiments, contacting the population of cells with the test agent can comprise culturing the population of cells in the presence of culture media flowing through the device.

In various embodiments, the first population of cells or the second population of cells, or both can be patient specific and the method models patient-specific parameters.

Various embodiments of the present invention provide for a method of producing a device of the present invention, comprising: seeding the first population of cells on one surface of the membrane in the device; and optionally, seeding second population of cells on the other surface of the membrane in the device; OR seeding the first population of cells in one chamber in the device; and optionally, seeding the second population of cells in the other chamber in the device.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1C show an overview of iPSC-derived Pancreatic Ductal differentiation in accordance with various embodiments of the present invention. 1A. Pancreatic Endoderm Protocol: Induced Pluripotent Stem Cells (IPSC) undergo differentiation via definitive endoderm (DE) germ layer, posterior foregut (PFG), pancreatic progenitors, toward pancreatic endoderm for a total of 13 days. RT-qPCR data of ductal genes to identify range of key time points for determining bipotent trunk phase (BTP). 1B. 2 days vs 4 days of Phase III: Shortening the endocrine protocol to 2 days followed by ductal specification increases ductal markers and decreases endocrine marker NKX6.1. 1C. iPan Ductal Specification. Signaling pathways involved in directing bipotent trunk progenitors to pancreatic ductal cells. Data is shown as mean±SEM with statistical significance determined by unpaired two-tailed t-test. *p<0.05, **p<0.01, ***p<0.001, ****p<0.0001. RNA was extracted from 3 IPSC lines, 7iCTR-n7, 3iCTR-n14 and Edi028, and qPCR was performed with 3-4 technical replicates. For FIG. 1A, all conditions were normalized to Day 0 and for FIG. 1B, 4 days of Phase III was normalized to 2 days.

FIGS. 2A-2B show terminal maturation for iPan Ductal cells in accordance with various embodiments of the present invention. 2A. Gene expression of ductal markers of samples differentiated with dissociation (4 bars to the right) or without dissociation (4 bars to the left, excluding iPSC bar), with or without addition of EGF, Noggin and Nicotinamide. Data is shown as mean±SEM with statistical significance determined by unpaired two-tailed t-test. *p<0.05, **p<0.01. qPCR was performed with 3 technical replicates. 2B. Differentiation schematic of IPSC-derived pancreatic ductal cells with key markers used for characterization.

FIGS. 3A-3B show iPan Ductal at Multiple Timepoints and in Various Formats in accordance with various embodiments of the present invention. 3A. Schematic preparation of cells dissociated and reseeded in multiple formats—planar, passaged planar (PP), organoid, organoid-to-planar (OTP) and collected at Day 21 and/or Day 31. Asterisks indicate single cell dissociation. 3B. Ductal gene expression in ductal cells cultured in various formats and at multiple timepoints—Day 21 and Day 31. Ductal samples were normalized to iPSC, and compared to Human Pancreatic Ductal Epithelium, HPDE6-E6E7, and Primary Human Exocrine. Data is shown as mean±SEM with statistical significance determined by unpaired two-tailed t-test. *p<0.05, **p<0.01, ***p<0.001, ****p<0.0001. RNA collected from iPSC line 03iCTRn14, 2-3 biological samples and 3 technical replicates.

FIGS. 4A-4D Functional Characterization of Planar iPan Ductal Cells. A. qPCR of Day 21 iPan Ductal in comparison to iPSCs and Human Pancreatic Ductal Epithelium, HPDE6-E6E7. Data is shown as mean±SEM with statistical significance determined by unpaired two-tailed t-test. *p<0.05, **p<0.01, ***p<0.001, ****p<0.0001. Black stars show significant upregulation from iPSCs, while red stars show significant downregulation from iPSCs. B. ICC of Day 21 cells show ductal markers i. SOX9 (green), HNF1B (grey), CK19 (red), ii. SOX9 (green), HNF1B (grey), chloride transporter CFTR (red), iii. sodium transporter ENAC (green), epithelial ECAD (grey), CFTR (red). Scale bar shown is 100 μM. C. Functional Iodide Efflux Assay of Day 29 iPan Ductal Cells. Graph shows concentration of accumulated iodide collected at 1-minute intervals for 8 minutes after the addition of NaNO3 with or without 10 μM Forskolin. Representative data from 3 lines (Edi028, 7iCTR-n7 and 3iCTR-n14). D. Western Blot of samples used for Iodide Efflux Assay after 2 rounds of 15 minute exposure to Forskolin 0 hr and 24 hr. Samples show protein expression of ductal markers (SOX9, CK19, CA2, CFTR) and phosphorylated proteins involved in Forskolin induced cAMP signaling pathway (p-CREB, p-ATF1) as well as non-phosphorylated CREB. HMGB1 was used as loading control.

FIGS. 5A-5C show Characterization of Functional Organoid iPan Ductal Cells in accordance with various embodiments of the present invention. 5A. qPCR of Day 21, 29 and 35 Organoids in comparison to HPDE6-E6E7 Organoids. Data is shown as mean±SEM with statistical significance determined by unpaired two-tailed t-test. *p<0.05, **p<0.01, ***p<0.001, ****p<0.0001. RNA collected from iPSC line Edi-028, pooled from 4-12 wells of a 12-w plate. qPCR performed with 3 technical replicates. 5B. ICC of Day 30 organoids. i. ICC of sectioned organoids show eCad (green, left panel), sodium transporter ENAC (green, middle panel), CFTR (red, left panel), cytoskeletal CK19 (red, middle and right panel), ii. ICC of whole mount organoids show SOX9 (green), eCad (red, left 2 panels), CK19 (red, right 2 panels) at Z-planes Z=1 (top of organoid) and Z=0 (center of organoid). Scale bar shown is 100 μM. 5C. Forskolin Induced Swelling assay show functional organoids. i. Brightfield images of organoids were taken before and after Forskolin treatment (10 μM) for 2 hours. Distance x (diameter of lumen) and distance y (diameter of organoid) were manually measured with ImageJ then converted to volume. ii. Graph shows ratio of lumen volume to organoid volume for control and forskolin conditions respectively. 52 organoids from 2 iPSC lines (Edi-028 and 03iCTR-n14) were measured. Data is shown as mean±SEM with statistical significance determined by unpaired two-tailed t-test. ****p<0.0001.

FIGS. 6A-6D show Characterization of Functional iPan Ductal CHIPs in accordance with various embodiments of the present invention. 6A. Schematic iPan Ductal CHIP Differentiation. On Day 7, cells were seeded in Chips then on Day 8, flow was initiated at 30 μL/hr for dynamic system or kept stagnant. 6B. ICC of Day 21 static CHIP. ICC show SOX9 (green), CK19 (red) at Z-planes 2 (top of PDMS membrane) and 3 (bottom of PDMS membrane). Scale bar shown is 200 uM. 6C. ICC of Day 21 dynamic CHIP. ICC show ductal markers SOX9 (green), HNF1B (grey), chloride transporter CFTR (red, left panel), ZO1 (red, middle panel), CK19 (red, right panel). Scale bar shown is 100 μM. 6D. Functional Iodide Efflux Assay of iPan Ductal CHIP. Graph shows concentration of iodide collected at Period A for 3 minutes and Period B for 8 minutes after the addition of NaNO3 with or without 10 μM Forskolin.

FIG. 7 depicts Initial attempt at Ductal Differentiation From Pancreatic Endoderm in accordance with various embodiments of the present invention. Cells exhibit pluripotency markers—OCT4 (green) and SSEA4 (red) on Day 0, and Definite Endoderm markers SOX17 (green) and FOXA2 (red) on Day 4. After 4 days of Phase Ill and 4 days of Phase IV, cells show Pancreatic Endoderm markers SOX9 (green) and NKX6.1 (red). After 2 weeks of Phase V with notch-activating ligand, sDLL1, Pancreatic Endocrine marker C-Peptide (green) is observed on Day 21. No ductal markers, CK19, observed at this stage. Scale bar is 50 μm.

FIG. 8 depicts Pancreatic Endoderm Differentiation From Thawed Pancreatic Progenitors in accordance with various embodiments of the present invention. top. Schematic of dissociating, freezing, and thawing of cells. middle. Thawed and fresh cells show Pancreatic Endoderm markers PDX1 (green) and NKX6.1 (red) on Day 13. Scale bar is 50 μm. bottom. Quantification of % PDX1⁺ NKX6.1⁺ cells of fresh and thawed cells. (n=27-36 sites, 3-4 technical replicates.)

FIG. 9A-9B depicts iPan Ductal Cells Show Versatility in Generating Adherent, Organoid or CHIP Cultures from Thawed Progenitors in accordance with various embodiments of the present invention. 9A. BTP cells seeded on a i. Matrigel-coated plate for planar culture; ii. in a Matrigel bubble for organoid culture; or iii. on top channel for CHIP culture. ICC shows ductal markers SOX9 (green) and CK19 (red) in all 3 formats. Scale bar shown is 100 μm. 9B. Organoid-To-Planar (OTP), iPan Ductal organoids seeded to planar. OTP culture shows ductal markers SOX9 (green), HNF1B (gray), CK19 (red, left panel), CFTR (red, right panel) and tight junction marker, ZO1 (red, middle panel). Scale bar shown is 100 μm.

FIG. 10 depicts Schematic Workflow of Functional Iodide Efflux Assay of Planar iPan Ductal Cells in accordance with various embodiments of the present invention. Supernatant collected at 1-minute intervals for 8 minutes after the addition of NaNO3 with or without 10 μM Forskolin. Aliquots collected were measured with iodide electrode.

FIG. 11 depicts Carbonic Anhydrase Activity of Day 29 iPan Ductal cells in Planar, Organoid and OTP Formats in accordance with various embodiments of the present invention. iPan Ductal cells in comparison to iPSC, Human Pancreatic Ductal Epithelium HPDE6-E6E7, and Primary Human Exocrine tissue from City of Hope. CA activity was measured with colorimetric assay and normalized to protein concentration (BCA assay). Data is shown as mean ±SEM with statistical significance determined by unpaired two-tailed t-test. *p<0.05, **p<0.01.

FIG. 12 depicts iPan Ductal Organoids Dissociated Then Seeded To Transwells, Organoid-To-Transwells (OTT) in accordance with various embodiments of the present invention. 12A. Single cell dissociated Day 21 organoids seeded on transwells and cultured up to Day 29 or longer. 12B. Schematic of a single transwells system (of a 24-well plate), indicating the porous membrane as Z=0 (basal) and the top of the cells as Z=1 (apical). C. OTT culture shows DAPI (blue) and ductal marker CFTR (red). Scale bar is 100 μm.

FIG. 13 depicts iPan ductal cells show emergence of mature ductal markers in function of time in accordance with various embodiments of the present invention.

FIG. 14A-14D depicts timing optimization for initiation of pancreatic acinar differentiation from hiPSC-generated pancreatic progenitors in accordance with various embodiments of the present invention. 14A: Schematic of differentiation process; 14B: qPCR deciding to start acinar differentiation after 4 days of stage 3; 14C: ICC images; 14D: Histogram of ICC quants.

FIG. 15 depicts modulation of FGF, Wnt and Notch signaling contribute to early acinar cell fate determination in accordance with various embodiments of the present invention. Top: Modulating Notch signaling in the presence of FGF10 has a significant impact in improving the efficiency of the percentage of amylase positive acinar cells that can derived from iPSCs. Treatment with XXI, a Notch signaling inhibitor, since it has the most increase in amylase positive clusters and lowest PDX1 levels, while treatment with sDLL1 presence of the Notch receptor-activating ligand, soluble Delta Like Canonical Notch Ligand 1 (sDLL1) does not increase the Amylase positive clusters or decrease PDX1 levels. FGF10 is added to all conditions. Bottom: FGF, Wnt3a, XXI, E176; 20 ng/ml FGF10 combined with Wnt3A and XXI shows best amylase expression indicative of the optimal condition for acinar cell specification

FIG. 16 depicts nicotinamide addition, BMP signaling antagonism plays key role in acinar differentiation in accordance with various embodiments of the present invention.

FIGS. 17A-17E depicts organoid generation of acini in accordance with various embodiments of the present invention. 17A. Schematic; 17B. Brightfield images of generated acini organoids; 17C-17E. Immunohistochemistry of sectioned organoids depicting expression of acinar-specific and epithelial cell proteins in a 3D organoid structure.

FIG. 18 shows that inducing PTF1A expression in iPSC-derived pancreatic progenitors using lentiviral vector improved Acinar specification as can be seen expression of Acinar-specific genes.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein the term “organ chip” (also referred to as “organ on chip”) refers to a microfluidic culture device are capable of recapitulating the microarchitecture and functions of living organs

Described herein, our invention presents a model of diseases and of the pancreas itself which offers the ability to observe real time changes. We can recapitulate in this in multiple formats which allow for live cell assays and for each patient in a chip format. This is difficult to model in animals since pancreatitis patients, for example, have a complex set of polygenic mutations. Using patient iPSC-derived pancreatic exocrine cells on a tissue-chip we can replay the disease process ex vivo and discover new class of drugs that can treat the disease early during progression. In addition, our model is not as time sensitive or resource-limited as primary cell culture, while still being a human cell model as opposed to an animal cell model. Finally, our use of iPSCs allows for patient-specific studies.

Our lab has successfully developed a process for differentiating mature and functional pancreatic ductal cells from iPSCs. The ductal differentiation protocol has been improved further with knowledge gained from the development of pancreatic islet differentiation protocols to better mimic embryonic development of pancreatic ductal epithelium. Terminally differentiated pancreatic endocrine and pancreatic acinar and ductal cells emerge at different points during transition through the pancreatic progenitor phase during embryonic pancreatic development. The tip further develops to acinar cells, while the trunk gives rise to pancreatic ductal and/or endocrine cells. Notch signaling promotes differentiation towards a ductal fate while Notch inhibition promotes pancreatic endocrine specification. Therefore, the terminal maturation phase includes supplementing the media with a soluble DLL1, which is a Notch receptor pathway modulating ligand.

We have shown that pancreatic progenitors can be frozen and thawed for reliable iPan Ductal differentiation across multiple 2D and 3D culture formats—planar, organoid, Transwell and Tissue chips.

There is utility in establishing a differentiation protocol in multiple formats as each format has its own advantages in terms of applications. Therefore, this gives us the freedom to explore different approaches to investigations of ductal-differentiated cells in various pancreatic disease models. Our iPan Ductal cells can be used for multiple applications. The planar 2D format is ideal for studies requiring testing of numerous potential drugs and pathway regulators in a high-throughput screen. The effects of many treatments can be efficiently screened in 96-, 384-, 1536 well plates. By such an approach, we can test for thousands of compounds that can potentially remediate any loss of CFTR protein Nevertheless, static planar cultures may not always accurately recapitulate exocrine pancreatic disease pathophysiology, as they do not promote formation of three-dimensional structures and thus maybe inadequate for performing certain functional assays. We have successfully modeled growth of pancreatic exocrine cells in organoids, Transwells and a dynamic Tissue-chip system that allows cells to polarize and better mimic human ductal biology architecturally and functionally.

Forskolin-induced swelling has been previously used as an assay to measure the function of CFTR, an ion channel located in the apical membrane of pancreatic ductal epithelium cells. CFTR mediates the transport of water and chloride regulated via cAMP signaling, and organoids with a lumen swell when forskolin, an activator of adenylyl cyclase is introduced.

This phenomenon cannot not be visually observed in planar cultures due to the lack of polarization of the membrane since the cells do not aggregate to form a closed structure with a lumen.

Here, we demonstrated growth of organoids in 96-well plates with potential applicability for high throughput screening studies to screen potential CFTR correctors or other compounds. We have defined a new set of minimal conditions for bulk production of such organoid cultures, which we have also analyzed by other assays including immunocytochemistry of sectioned organoids. Most common CFTR mutations have been categorized into 6 classes and potential drugs have been used in trials to target each class. Our 3D model system would be an effective way to test whether these drugs improve CFTR function, as measured, e.g., by organoid swelling. Recently, Dekkers et al reported that in vitro swelling of organoids obtained from rectal epithelium of CP patients can be correlated to clinical biomarkers such as sweat chloride concentration measurements, which may help to identify subjects with CF who will respond well to CFTR-targeted drugs. Our procedure for reprogramming patient peripheral blood mononuclear cells to iPSCs and further specifying iPan Ductal cells may also enable us to select the most efficacious drugs for each CP patient.

The dynamic chip platform (tissue-chip (“organ-on-chip”) technologies) is especially useful tool to study CFTR function. By flushing media at a flow rate like that in the human pancreatic ducts, changes in pH levels and bicarbonate and chloride ions can be measured in the trans-epithelial conditioned media. In such a platform, which incorporates distinct chambers separated by semipermeable artificial membranes. These systems also allow for iPan ductal to be experimentally co-cultured together with other relevant cell types such as pancreatic acinar, endocrine, stellate, and/or endothelial cells, allowing close contact and providing for crucial interchanges of biochemical information between cell types. Kyu Shik et al isolated primary pancreatic ductal tissue by micro dissecting patient-derived human pancreatic ducts from pancreatic remnant cell pellets and then co-cultured pancreatic ductal epithelium (with mutated CFTR) with islets. However, given that CP is a complex disease and obtaining primary patent-derived ductal tissue from a range of patient genotypes is not a scalable approach, our iPSC platform described here allows us to circumvent some of these issues. Responses to other cells in the system can potentially be measured from secretions; and/or changes in gene expression in a particular cell type. We believe that some of the dynamic properties of in vivo systems may be possible to mimic using co-cultures that combine distinct cell types grown in separate compartments, thereby allowing experiments such as adding drug compounds or secretagogues to the apical side (top channel) and collecting effluent from the basal side (bottom channel) to measure iodide, pH, and bicarbonate levels.

Overall, we have delineated a simple and reliable method to differentiate iPSC-derived pancreatic ductal cells from start to finish that is applicable across different patient-derived iPSC lines and demonstrated their adaptation to multiple 2D and 3D cell culture platforms including planar cultures, organoids, Transwell and Tissue-chips. Our clean workflow of procedures incorporates steps for characterizing distinct stages and functional assays to test for ductal cell behaviors in healthy and disease settings. We have reprogrammed iPSCs from a range of chronic pancreatitis patients both with and without specific genetic alterations related to hereditary CP and expect to further explore the disease pathophysiology in future studies using these 2D and 3D cell formats developed here.

Impacts of monogenic and polygenic genetic variants associated with pancreatitis patient-specific iPSC lines can be assessed by utilizing the described platform. Ultimately, the ductal disease of iPan Ductal cells can be rescued by high-throughput screening for identifying novel therapeutics.

Thus, this novel platform can be now applied to study pancreatic ductal diseases and to discover and develop novel treatments.

We have also developed novel protocols for production of acinar cells, which show functionality in both adherent and organoid formats.

We have determined that EGF, FGF (FGF10) and Notch signaling pathway modulation (specifically adding sDLL1) after formation of definitive endoderm and then pancreatic endoderm drives pancreatic ductal cell fate from hiPSCs. We have determined that Wnt (Wnt3a), FGF (FGF10) and Notch signaling pathway modulation (specifically blocking Notch signaling with XXI) after formation of definitive endoderm and then pancreatic endoderm drives pancreatic acinar cell fate from hiPSCs. Further, addition of Noggin and Nicotinamide (promotes cell survival and differentiation) increased efficiency of acinar differentiation.

These differentiated cells can be used to model these diseases, and test gene therapy and other forms of treatment. This allow the study of the pancreatic exocrine development mechanisms, as well as the drivers of the diseases and potential cures. iPSC-generated cells also can be used for generating patient-specific treatments, because cell lines can be made for each patient.

Pancreatic ductal and endocrine cells develop from a pool of bipotent trunk progenitors where the bifurcation is mediated by notch signaling. Notch signaling promotes differentiation towards ductal fate while EGF signaling induces proliferation of ductal epithelial cells. Day 8 of differentiation indicates an emergence of SOX9⁺PDX1⁺ bipotent trunk cell population, at which point, activating high notch signaling pushes it towards ductal cells. Determining the temporal window of notch activation is critical as activating it too late could result in a higher ratio of C-Peptide⁺ endocrine cells.

In the past, optimization of experiments is slow and arduous as it takes a month long to expand and differentiate iPSCs plus an additional week to characterize cells. Therefore, the present methods are efficient and allows us to bulk differentiate and cryopreserve pancreatic progenitors. This also removes the factor of batch-to-batch differentiation variability and provides the flexibility to seed progenitors for planar or organoid differentiation towards pancreatic endocrine or exocrine lineage.

At day 11, midway through phase 3, some groups were single cell dissociated and reseeded at 30% confluency. Dissociation shows a significant decrease in GATA4 expression, a pancreatic progenitor and acinar marker. Also, dissociated cells with exposure to EGF show increased levels of ductal KRT19, HNF1B, SOX9, and FOXA2 markers. Western blot data exhibit increased levels of CK19 and PDX1 in all conditions from iPSC, but do not show distinguishing levels between the groups. Conditions with Noggin and Nicotinamide were tested to promote further development towards NKX6.1⁺ population but before developing to NGN3⁺ endocrine specified population. Since there were no apparent differences in conditions with or without noggin and nicotinamide, those 2 reagents were excluded. Based on results, iPan Ductal protocol includes dissociation and EGF signaling.

Prior to seeding, organ chips were plasma activated and MATRIGEL coated. The following day, Day 11 cells were dissociated and reseeded into the top channel at a 5 million cells/mL density in Phase 3 media. The bottom channel was filled with only Phase 3 media. 48 hours later, media was replaced with Phase 4 media. Feeding was done every other day for the next 2 weeks.

Accordingly, the present invention is derived, in part, by these findings.

Various embodiments of the present invention provide for a method of differentiating induced pluripotent stem cells (iPSCs) into iPSC-derived pancreatic progenitor cells, comprising: seeding induced pluripotent stem cells (iPSCs) on a solid medium coated with solubilized basement membrane preparation in the presence of serum-free, stabilized cell culture medium, and optionally a ROCK inhibitor; culturing the cells in a first culture medium comprising a first base medium Activin A, CHIR99021, and a ROCK Inhibitor; culturing the cells in a second culture medium comprising the first base medium and Activin A, and bFGF (FGF-2); culturing the cells in a third culture medium comprising the first base medium and FGF10, NOGGIN, and CHIRR99021; culturing the cells in a fourth culture medium comprising a second base medium and FGF10, NOGGIN, All-trans Retinoic Acid, and SANT1, wherein iPSC-derived pancreatic progenitor cells are produced.

In various embodiments, Activin A, CHIR99021, and the ROCK Inhibitor in the first culture medium is at a concentration of about 100 ng/ml Activin A, about 2 uM CHIR99021 and about 10 uM of the ROCK inhibitor. In various embodiments, Activin A, and bFGF (FGF-2) in the second culture medium is at a concentration of about 100 ng/ml Activin A and about 5 ng/ml bFGF (FGF-2). In various embodiments, FGF10, NOGGIN, and CHIRR99021 in the third culture medium is at a concentration of about 50 ng/ml FGF10, about 50 ng/ml NOGGIN and about 0.25 uM CHIRR99021. In various embodiments, FGF10, NOGGIN, All-trans Retinoic Acid, and SANT1 in the fourth culture medium is at a concentration of about 50 ng/ml FGF10, about 50 ng/ml NOGGIN, about 2 uM All-trans Retinoic Acid, and about 0.25 uM SANT1.

In various embodiments, the cells are cultured in the first culture medium for about 1 day. In various embodiments, the cells are cultured in the second culture medium for about 1 to 2 days. In various embodiments, the cells are cultured in the third culture medium for about 1 to 2 days. In various embodiments, the cells are cultured in the fourth culture medium for about 1 day.

Various embodiments provide for a method of differentiating induced pluripotent stem cells (iPSCs) into induced pancreatic (iPan) ductal cells, comprising seeding induced pluripotent stem cells (iPSCs) on a solid medium coated with solubilized basement membrane preparation in the presence of serum-free, stabilized cell culture medium, and optionally a ROCK inhibitor; culturing the cells in a first culture medium comprising a first base medium Activin A, CHIR99021, and a ROCK Inhibitor; culturing the cells in a second culture medium comprising the first base medium and Activin A, and bFGF (FGF-2); culturing the cells in a third culture medium comprising the first base medium and FGF10, NOGGIN, and CHIRR99021; culturing the cells in a fourth culture medium comprising a second base medium and FGF10, NOGGIN, All-trans Retinoic Acid, and SANT1; seeding the iPSC-derived pancreatic progenitor cells on a solid medium coated with solubilized basement membrane preparation in the presence of a fifth culture medium comprising a second base medium and a ROCK inhibitor; culturing the cells in a sixth culture medium comprising the second base medium and FGF10, EGF, and sDLL-1, wherein iPan ductal cells are produced.

In various embodiments, the method further comprises dissociating the iPSC-derived pancreatic progenitor cells before seeding the iPSC-derived pancreatic progenitor cells on the solid medium.

In various embodiments, the ROCK inhibitor in the fifth culture medium is at a concentration of about 10 uM. In various embodiments, the ROCK inhibitor in the fifth culture medium is at a concentration of about 5-15 uM.

In various embodiments, FGF10, EGF, and sDLL-1 in the sixth culture medium is at a concentration of about 25 ng/ml FGF10, about 50 ng/ml EGF, and about 50 ng/ml sDLL-1. In various embodiments, FGF10, EGF, and sDLL-1 in the sixth culture medium is at a concentration of about 20-30 ng/ml FGF10, about 40-60 ng/ml EGF, and about 40-60 ng/ml sDLL-1.

In various embodiments, the cells are cultured in the fifth culture medium for about 1 day. In various embodiments, the cells are cultured in the fifth culture medium for about 1-2 days. In various embodiments, the cells are cultured in the sixth culture medium for about 16 days. In various embodiments, the cells are cultured in the sixth culture medium for about 14-18 days.

Various embodiments provide for a method of dedifferentiating induced pluripotent stem cells (iPSCs) into induced pancreatic endocrine cells, comprising seeding induced pluripotent stem cells (iPSCs) on a solid medium coated with solubilized basement membrane preparation in the presence of serum-free, stabilized cell culture medium, and optionally a ROCK inhibitor; culturing the cells in a first culture medium comprising a first base medium Activin A, CHIR99021, and a ROCK Inhibitor; culturing the cells in a second culture medium comprising the first base medium and Activin A, and bFGF (FGF-2); culturing the cells in a third culture medium comprising the first base medium and FGF10, NOGGIN, and CHIRR99021; culturing the cells in a fourth culture medium comprising a second base medium and FGF10, NOGGIN, All-trans Retinoic Acid, and SANT1; seeding the iPSC-derived pancreatic progenitor cells on a solid medium coated with solubilized basement membrane preparation in the presence of a fifth culture medium comprising a second base medium and a ROCK inhibitor; culturing the cells in a sixth culture medium comprising the second base medium and Noggin, EGF and nicotinamide, wherein pancreatic endocrine cells are produced.

In various embodiments, the method further comprises dissociating the iPSC-derived pancreatic progenitor cells before seeding the iPSC-derived pancreatic progenitor cells on the solid medium.

In various embodiments, the ROCK inhibitor in the fifth culture medium is at a concentration of about 10 uM. In various embodiments, the ROCK inhibitor in the fifth culture medium is at a concentration of about 5-15 uM.

In various embodiments, Noggin, EGF and nicotinamide in the sixth culture medium is at a concentration of about 50 ng/ml Noggin, about 100 ng/ml EGF, and about 10 mM nicotinamide. In various embodiments, Noggin, EGF and nicotinamide in the sixth culture medium is at a concentration of about 40-60 ng/ml Noggin, about 75-125 ng/ml EGF, and about 5-15 mM nicotinamide.

In various embodiments, the cells are cultured in the fifth culture medium for about 4 days. In various embodiments, the cells are cultured in the fifth culture medium for about 3-5 days. In various embodiments, the cells are cultured in the sixth culture medium for about 4 days. In various embodiments, the cells are cultured in the sixth culture medium for about 3-5 days.

Various embodiments provide for a method of producing organoids comprising pancreatic ductal cells, comprising: seeding induced pluripotent stem cells (iPSCs) on a solid medium coated with solubilized basement membrane preparation in the presence of serum-free, stabilized cell culture medium, and optionally a ROCK inhibitor; culturing the cells in a first culture medium comprising a first base medium Activin A, CHIR99021, and a ROCK Inhibitor; culturing the cells in a second culture medium comprising the first base medium and Activin A, and bFGF (FGF-2); culturing the cells in a third culture medium comprising the first base medium and FGF10, NOGGIN, and CHIRR99021; culturing the cells in a fourth culture medium comprising a second base medium and FGF10, NOGGIN, All-trans Retinoic Acid, and SANT1 to produce iPSC-derived pancreatic progenitor cells; dissociated the iPSC-derived pancreatic progenitor cells; seeding the iPSC-derived pancreatic progenitor cells into a solubilized basement membrane preparation and plating the seeded cells onto a solid medium; and culturing the cells in a sixth culture medium comprising the second base medium and FGF10, EGF, and sDLL-1.

Various embodiments provide for a method of producing organoids comprising pancreatic ductal cells, comprising: providing dissociated iPSC-derived pancreatic progenitor cells; seeding the cells into a solubilized basement membrane preparation and plating the seeded cells onto a solid medium; and culturing the cells in a sixth culture medium comprising the second base medium and FGF10, EGF, and sDLL-1. In various embodiments the concentrations are: about 20-30 ng/ml FGF10, about 40-60 ng/ml EGF, and about 40-60 ng/ml sDLL-1. Various embodiments provide for a method of producing organoids comprising pancreatic ductal cells, comprising: providing dissociated iPSC-derived pancreatic progenitor cells; seeding the cells into a solubilized basement membrane preparation and plating the seeded cells onto a solid medium; and culturing the cells in a sixth culture medium comprising the second base medium and FGF10, EGF, and sDLL-1. In various embodiments the concentrations are: about 25 ng/ml FGF10, about 50 ng/ml EGF, and about 50 ng/ml sDLL-1. In various embodiments, the cells are fed with the sixth culture medium every 1-4 days for at least 2 weeks. In various embodiments, the cells are fed with the sixth culture medium every 2-3 days for at least 2 weeks. In various embodiments, the cells are cultured for about 2 weeks, about 2.5 weeks, about 3 weeks, about 4 weeks, about 5 weeks, about 6 weeks, about 7 weeks or about 8 weeks.

In various embodiments, the iPSC-derived pancreatic progenitor cells are resuspended in the solubilized basement membrane preparation at a density of about 2 million cells/mL. In various embodiments, the density is about 1-3 million cells/mL. In various embodiments, the plating the seeded cells onto a solid medium comprises plating about 10 μL of cell suspension onto each solid medium. In various embodiments, the plating the seeded cells onto a solid medium comprises plating about 5-15 μL of cell suspension onto each solid medium. In various embodiments, the solid medium is a U-bottom multi-well plate. In various embodiments, once the Matrigel dome is solidified, Phase III medium with Rho kinase Inhibitor was added for 24 h, then the medium was switched to Phase IV media and cultured for 2 weeks or longer, whilst feeding every 2 days.

For the second method of organoid culture, pancreatic progenitors (day 7) were single cell dissociated and resuspended in Matrigel at a density of 2 million cells/mL. 10 uL of cell suspension was seeded in each well of a 96 U-bottom plate then incubated at 37 C for 15 minutes flipped. Then cells were fed with 100 ul of ductal media with FGF10, EGF and sDLL1 every 2-3 days for at least 2 weeks. The second method of organoid culture favor more ductal organoids Concentrations are: 25 ng/ml FGF10, about 50 ng/ml EGF, and about 50 ng/ml sDLL-1. In various embodiments, cells were fed with about 50-150 ul of ductal media with FGF10, EGF and sDLL1 every 2-3 days for at least 2 weeks; and the second method of organoid culture favor more ductal organoids Concentrations are: about 20-30 ng/ml FGF10, about 40-60 ng/ml EGF, and about 40-60 ng/ml sDLL-1.

Various embodiments of the present invention provide for a method of differentiating induced pluripotent stem cells (iPSCs) into iPSC derived pancreatic acinar cells, comprising: seeding induced pluripotent stem cells (iPSCs) on a solid medium coated with solubilized basement membrane preparation in the presence of serum-free, stabilized cell culture medium, and optionally a ROCK inhibitor; culturing the cells in a first culture medium comprising a first base medium Activin A, CHIR99021, and a ROCK Inhibitor; culturing the cells in a second culture medium comprising the first base medium and Activin A, and bFGF (FGF-2); culturing the cells in a third culture medium comprising the first base medium and FGF10, NOGGIN, and CHIRR99021; culturing the cells in a fourth culture medium comprising a second base medium and FGF10, NOGGIN, All-trans Retinoic Acid, and SANT1 to produce iPSC-derived pancreatic progenitor cells; seeding the iPSC-derived pancreatic progenitor cells into a solubilized basement membrane preparation and plating the seeded cells onto a solid medium; culturing the cells in a seventh culture medium comprising a second base medium and XXI, FGF10, Noggin, Nicotinamide, and Murine Wnt3a, wherein iPSC derived pancreatic acinar cells are produced.

In various embodiments, XXI, FGF10, Noggin, Nicotinamide, and Murine Wnt3a in the seventh culture medium is at a concentration of about 20 ng/mL FGF10, 25 ng/mL Wnt3a, 1 uM XXI, 50 ng/mL Noggin, and 10 mM Nicotinamide. In various embodiments, XXI, FGF10, Noggin, Nicotinamide, and Murine Wnt3a in the seventh culture medium is at a concentration of about 15-25 ng/mL FGF10, 20-30 ng/mL Wnt3a, 0.1-1.5 uM XXI, 40-60 ng/mL Noggin, and 5-15 mM Nicotinamide.

In various embodiments, differentiating induced pluripotent stem cells (iPSCs) into iPSC derived pancreatic acinar cells further comprises inducing overexpression of acinar specific genes in the iPSC-derived pancreatic progenitor cells. In various embodiments, the acinar specific genes are selected from PTF1A, XBP1, RBPJL, MIST1, GATA4, and combinations thereof. In various embodiments, inducing overexpression of acinar specific genes in the iPSC-derived pancreatic progenitor cells can comprise inducing the expression when the cells were iPSCs. In various embodiments, inducing overexpression of acinar specific genes in the iPSC-derived pancreatic progenitor cells can comprise inducing the expression when the cells are pancreatic progenitor cells (e.g., bipotent trunk progenitors).

In various embodiments, inducing overexpression of acinar specific genes in the iPSC-derived pancreatic progenitor cells comprises transducing the iPSCs derived pancreatic progenitor cells with a vector comprising a nucleic acid encoding the acinar specific genes. In various embodiments, inducing overexpression of acinar specific genes in the iPSC-derived pancreatic progenitor cells comprise transducing the iPSCs derived pancreatic progenitor cells with a vector comprising a nucleic acid encoding the acinar specific genes. In various embodiment, both can be done. In various embodiments, the vector is a lentivirus.

In various embodiments, differentiating induced pluripotent stem cells (iPSCs) into iPSC derived pancreatic acinar cells further comprises inducing PTF1A expression in the iPSC-derived pancreatic progenitor cells. In various embodiments, inducing PTF1A expression in the iPSC-derived pancreatic progenitor cells can comprise inducing the expression when the cells were iPSCs. In various embodiments, inducing PTF1A expression in the iPSC-derived pancreatic progenitor can comprise inducing the expression when the cells are pancreatic progenitor cells. In various embodiment, both can be done.

In various embodiments, inducing PTF1A expression in the iPSC-derived pancreatic progenitor cells comprises transducing the iPSCs derived pancreatic progenitor cells with a vector comprising a nucleic acid encoding PTF1A. In various embodiment, both can be done. In various embodiments, inducing PTF1A expression in the iPSC-derived pancreatic progenitor cells comprise transducing the iPSCs derived pancreatic progenitor cells with a vector comprising a nucleic acid encoding PTF1A. In various embodiment, both can be done.

In various embodiments, inducing PTF1A expression in the iPSC-derived pancreatic progenitor cells comprises transducing the iPSCs with a lentivirus vector comprising a nucleic acid encoding PTF1A. In various embodiments, inducing PTF1A expression in the iPSC-derived pancreatic progenitor cells comprise transducing the iPSCs derived pancreatic progenitor cells with a lentivirus vector comprising a nucleic acid encoding PTF1A. In various embodiment, both can be done.

Various embodiments provide for a method of producing organoids comprising pancreatic acinar cells, comprising: seeding induced pluripotent stem cells (iPSCs) on a solid medium coated with solubilized basement membrane preparation in the presence of serum-free, stabilized cell culture medium, and optionally a ROCK inhibitor; culturing the cells in a first culture medium comprising a first base medium Activin A, CHIR99021, and a ROCK Inhibitor; culturing the cells in a second culture medium comprising the first base medium and Activin A, and bFGF (FGF-2); culturing the cells in a third culture medium comprising the first base medium and FGF10, NOGGIN, and CHIRR99021; culturing the cells in a fourth culture medium comprising a second base medium and FGF10, NOGGIN, All-trans Retinoic Acid, and SANT1 to produce iPSC-derived pancreatic progenitor cells; culturing the cells in a seventh culture medium comprising a second base medium and XXI, FGF10, Noggin, Nicotinamide, and Murine Wnt3a, wherein iPSC derived pancreatic acinar cells are produced; dissociating the iPSC derived pancreatic acinar cells; seeding the iPSC-derived pancreatic acinar cells into a solubilized basement membrane preparation and plating the seeded cells onto a solid medium; and culturing the cells in an eighth culture medium comprising a second base medium and FGF10, Noggin, Nicotinamide, and Murine Wnt3a.

Various embodiments provide for a method of producing organoids comprising pancreatic acinar cells, comprising: providing dissociated iPSC derived pancreatic acinar cells; seeding the iPSC-derived pancreatic acinar cells into a solubilized basement membrane preparation and plating the seeded cells onto a solid medium; and culturing the cells in an eighth culture medium comprising a second base medium and FGF10, Noggin, Nicotinamide, and Murine Wnt3a.

In various embodiments, seeding the iPSC-derived pancreatic acinar cells comprises seeding at a concentration of about 1-3×10\hat{ }6 cells/about 1040 μL of the solubilized basement membrane preparation (e.g., MATRIGEL). The solubilized basement membrane preparation-cell mix is then aliquoted as about 15 μL/well of 96 well plate. In various embodiments, plating the seeded cells onto a solid medium comprises plating in about 10-20 μL bubbles and allowing the plated cells to sit for about 10-20 minutes prior to culturing the cells in the eighth culture medium. In various embodiments, plating the seeded cells onto a solid medium comprises plating in about 15 μL bubbles and allowing the plated cells to sit for about 15 minutes prior to culturing the cells in the eighth culture medium. In various embodiments, the cells are cultured in the eighth culture medium for about 40-48 days, and given the eighth culture medium about every day, every other day or every three days. In various embodiments, the cells are cultured in the eighth culture medium for about 44 days and given the eighth culture medium about every other day. In various embodiments, the solid medium is a round-bottom multi-well plate.

In various embodiments of these methods, the designation of first culture medium, second culture medium, third culture medium, fourth culture medium, fifth culture medium, sixth culture medium, seventh culture medium, either culture medium, or ninth culture medium does not denote the order of the culture media, or that certain methods must have all the culture media, or that certain methods must have a sequential number of culture media. This is merely used to easily identify and refer back to the different culture media. Similarly, the designation of a first base medium and a second base medium does not denote the order of the base media used in the methods. This is also merely used to easily identify and refer back to the different base media.

In various embodiments of these methods, the solubilized basement membrane preparation is extracted from the Engelbreth-Holm-Swarm (EHS) mouse sarcoma. In various embodiments, solubilized basement membrane preparation is MATRIGEL matrix.

In various embodiments of these methods, the ROCK inhibitor is Y27632 ROCK Inhibitor.

In various embodiments of these methods, the serum-free, stabilized cell culture medium is mTeSR™ plus medium.

In various embodiments of these methods, the first base medium comprises an agent from the group consisting of MCDB 131, Glutamax, Vitamin C, BSA, sodium bicarbonate, antibiotic, and combinations thereof. In various embodiments of these methods, the first base medium comprises an agent from the group consisting of MCDB 131, Glutamax, Vitamin C, BSA, sodium bicarbonate, antibiotic (e.g., PSA), and combinations thereof. In various embodiments, the first base medium comprises 2, 3, 4, 5, or 6 agents from the group consisting of MCDB 131, Glutamax, Vitamin C, BSA, sodium bicarbonate, antibiotic, and combinations thereof. In various embodiments, the first base medium comprises MCDB 131, Glutamax, Vitamin C, BSA, sodium bicarbonate, and antibiotic (e.g., PSA). In various embodiments, the first base medium comprises 5-6 agents from the group consisting of MCDB 131, Glutamax, Vitamin C, BSA, sodium bicarbonate, antibiotic (e.g., PSA), and combinations thereof. In various embodiments, the first base medium comprises MCDB 131, Glutamax, Vitamin C, BSA, sodium bicarbonate, and antibiotic (e.g., PSA). In various embodiments, the first base medium comprises 7 agents from the group consisting of MCDB 131, Glutamax, PSA, Vitamin C, BSA, sodium bicarbonate, antibiotic, and combinations thereof. In various embodiments, the first base medium comprises 6 agents from the group consisting of MCDB 131, Glutamax, Vitamin C, BSA, sodium bicarbonate, antibiotic, and combinations thereof. In various embodiments, the first base medium comprises MCDB 131, Glutamax, PSA, Vitamin C, BSA, sodium bicarbonate, and antibiotic. In various embodiments, the first base medium comprises MCDB 131, Glutamax, Vitamin C, BSA, sodium bicarbonate, and antibiotic.

In various embodiments of these methods, the Glutamax, PSA, Vitamin C, BSA, sodium bicarbonate, and antibiotic are at a concentration of about 1×Glutamax, about 1×Antibiotic, about 0.5% Bovine Serum Albumin, about 1.5 mg/ml Sodium Bicarbonate and about 250 uM Vitamin C. In various embodiments of these methods, the Glutamax, PSA, Vitamin C, BSA, sodium bicarbonate, and antibiotic are at a concentration of about 1×Glutamax, about 1×Antibiotic, about 0.25-0.75% Bovine Serum Albumin, about 1.0-2.0 mg/ml Sodium Bicarbonate and about 200-300 uM Vitamin C.

In various embodiments of these methods, the second base medium comprises an agent from the group consisting of DMEM, antibiotic, B27 without vitamin A, vitamin C and combination thereof. In various embodiments, the second base medium comprises 2, 3, or 4 agents from the group consisting of DMEM, antibiotic, B27 without vitamin A, vitamin C and combination thereof. In various embodiments, the second base medium comprises 2-3 agents from the group consisting of DMEM, antibiotic, B27 without vitamin A, vitamin C and combination thereof. In various embodiments, the second base medium comprises 4 agents from the group consisting of DMEM, antibiotic, B27 without vitamin A, vitamin C and combination thereof. In various embodiments, the second base medium comprises an agent from the group consisting of DMEM, antibiotic, B27 without vitamin A, and vitamin C.

In various embodiments of these methods, the antibiotic, B27 without vitamin A, and uM vitamin C are at a concentration of about 1X antibiotic, about 1X B27 without vitamin A, and about 250 uM vitamin C. In various embodiments of these methods, the antibiotic, B27 without vitamin A, and uM vitamin C are at a concentration of about 0.5-1.5×antibiotic, about 0.5-1.5×B27 without vitamin A, and about 200-300 uM vitamin C.

In various embodiments of these methods, the antibiotic is a Pen-Strep Antibiotic.

Various embodiments provide for an induced pluripotent stem cell (iPSC) derived pancreatic progenitor cell made by a method of the present invention.

Various embodiments provide for an induced pluripotent stem cell (iPSC) derived iPan ductal cell made by a method of the present invention.

Various embodiments provide for an induced pluripotent stem cell (iPSC) derived acinar cell made by a method of the present invention.

Various embodiments provide for an induced pluripotent stem cell (iPSC) derived pancreatic organoid comprising pancreatic acinar cells made by a method of the present invention.

Various embodiments provide for an induced pluripotent stem cell (iPSC) derived pancreatic organoid comprising pancreatic ductal cells made by a method of the present invention.

### Cell, Organoid and Organ Chip Models and Methods of Uses

Various embodiments of the present invention provide for a model, comprising a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; and a fluidic device, or a cell culture plate, or a multi-well culture plate, wherein the test agent and the population of cells are in contact in a fluidic device the cell culture plate, or the multi-well culture plate.

In various embodiments, the fluidic device, the cell culture plate, or the multi-well culture plate is a Transwell system. In various embodiments, the fluidic device is a microfluidic device. In various embodiments, the microfluidic device is an organ chip.

Various embodiments provide for a device, comprising: a membrane comprising a top surface and a bottom surface; a first channel in fluidic communication with the top surface of the membrane; a second channel in fluidic communication with the bottom surface of the membrane, wherein the first and second channels each comprises a surface that is parallel to the membrane; a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells in the first channel or the second channel; and optionally, a second population of cells in the first channel or the second channel, wherein the first population of cells and the second population of cells are in different channels, and wherein the first population of cells and the second population of cells are not the same type of cells. In various embodiments, the device comprises the second population of cells.

In various embodiments, wherein the device has the second population of cells, the second population of cells are selected from the group consisting of endocrine cells, stellate cells endothelial cells, pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells.

In various embodiments, the device further comprises at least one inlet port adapted for fluid entering the at least one inlet port; and at least one outlet port adapted for fluid exiting the at least one outlet port.

In various embodiments, the first and second channels comprise polydimethylciloxane. In various embodiments, the first channel and the second channel are microfluidic channels.

In various embodiments, the device further comprises one or more gels and the population of cells having been seeded on top of or into the one or more gels.

In various embodiments, the first population of cells, or the second population of cells or both are patient specific. In various embodiments, the first population of cells, or the second population of cells or both express a fluorescent reporter.

Various embodiments provide for a device, comprising: a top chamber; a bottom chamber; a membrane between the top chamber and the bottom chamber; a first channel fluidically coupled to the top chamber; a second channel fluidically coupled to the bottom chamber; a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells in the first channel or the second channel; and optionally, a second population of cells in the first channel or the second channel, wherein the first population of cells and second population of cells are in different channels, and wherein the first population of cells and the second population of cells are not the same type of cells. In various embodiments, the device comprises the second population of cells.

In embodiments wherein the device comprises the second population of cells, the second population of cells are selected from the group consisting of endocrine cells, stellate cells endothelial cells, pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells.

In various embodiments, the first and second channels comprise polydimethylciloxane. In various embodiments, the first channel and the second channel are microfluidic channels.

In various embodiments, the device further comprises one or more gels and the population of cells having been seeded on top of or into the one or more gels.

In various embodiments, the first population of cells, or the second population of cells or both are patient specific. In various embodiments, the first population of cells, or the second population of cells or both express a fluorescent reporter.

Various embodiments of the invention provide for an organ chip device, comprising: a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells; optionally, a second population of cells; and a membrane separating the first population of cells and the second population of cells, wherein the first population of cells and the second population of cells are not the same type of cells. In various embodiments, the organ chip device comprises the second population of cells.

In various embodiments, the membrane comprises polydimethylciloxane.

In various embodiments, the organ chip device further comprises one or more gels and the population of cells having been seeded on top of or into the one or more gels.

In various embodiments, the first population of cells, or the second population of cells or both are patient specific. In various embodiments, the first population of cells, or the second population of cells or both express a fluorescent reporter.

Various embodiments of the present invention provide for a method of assessing a test agent, comprising: contacting the test agent to a device of the present invention as described herein, wherein the device comprises a population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells, and optionally, a second population of cells; measuring a parameter; and assessing the test agent based on the measured parameter.

In various embodiments, the device further comprises features as described herein; for example, a membrane comprising a top surface and a bottom surface, a first channel in fluidic communication with the top surface of the membrane, a second channel in fluidic communication with the bottom surface of the membrane, wherein the first and second channels each comprises a surface that is parallel to the membrane; or a top chamber, a bottom chamber, a membrane between the top chamber and the bottom chamber, a first channel fluidically coupled to the top chamber; a second channel fluidically coupled to the bottom chamber; or a membrane separating the first population of cells and the second population of cells,

In various embodiments, the first population of cells, or the second population of cells or both are patient specific. In various embodiments, the first population of cells, or the second population of cells or both express a fluorescent reporter.

In various embodiments, the test agent is a potential drug. In various embodiments, the test agent is a potential pathway regulator. In various embodiments, the test agent is a compound that can potentially improve CFTR function; for example, when tested with iPan Ductal cells from Chronic Pancreatitis patients with CFTR mutations. In various embodiments, the test agent is a chemotherapeutic agent. In various embodiments, the test agent is an infectious agent.

In various embodiments, measuring the parameter comprises measuring a phenotype of interest, expression level of a gene of interest, or expression level of a protein of interest, or combinations thereof. In various embodiments, measuring the parameter comprises measuring fluorescence. In various embodiments, measuring the parameter comprises measuring cell viability. In various embodiments, the parameter that is measured is iodide, pH levels and or bicarbonate levels.

In various embodiments, contacting the population of cells with the test agent comprises culturing the population of cells in the presence of culture media flowing through the device.

In various embodiments, the first population of cells or the second population of cells, or both are patient specific and the method models patient-specific parameters.

Various embodiments of the present invention provide for a method of assessing a test agent, comprising: contacting the test agent to a population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells, and assessing the test agent based on the measured parameter.

Various embodiments of the present invention provide for a method of assessing a test agent, comprising: contacting the test agent to a population of cells selected from the group consisting of pancreatic ductal cells derived from iPSCs, pancreatic ductal cells derived from pancreatic progenitor cells that were previously derived from induced pluripotent stem cells (iPSCs), pancreatic acinar cells derived from iPSCs, and pancreatic acinar cells derived from pancreatic progenitor cells that were previously derived from induced pluripotent stem cells (iPSCs), and assessing the test agent based on the measured parameter.

In various embodiment, the population of cells are arranged in a planar configuration. In various embodiments, the population of cells are arranged as an organoid. In various embodiments, the population of cells are in a Transwell system. In various embodiments, the population of cells are in a multiwall plate.

Various embodiments of the present invention provide for a method of producing a device of the present invention, comprising: seeding the first population of cells on one surface of the membrane in the device; and optionally, seeding second population of cells on the other surface of the membrane in the device.

Various embodiments of the present invention provide for a method of producing a device of the present invention, comprising: seeding the first population of cells in one chamber in the device; and optionally, seeding the second population of cells in the other chamber in the device.

In various embodiments, prior to seeding, chips were plasma activated and Matrigel coated. The following day, Day 7 cells were dissociated and reseeded into the top channel at a density of about 10 million cells/mL in Phase III media plus about 10 μM Rho kinase Inhibitor. The bottom channel was filled with only Phase III media plus about 10 Rho kinase Inhibitor. 24 hours later, media was replaced with Phase IV media. For Chips in dynamic system, Chips were placed in ZOE and a flow rate of about 30 μl/hr was initiated 48 hours post-seeding. Feeding was done every other day for the next 2 weeks.

In various embodiments, when organoids are seeded to Transwells or planar, organoids were incubated in Cell Recovery Solution (Corning) for an hour in 4° to remove Matrigel, then washed 3× with PBS. Organoids were then single cell dissociated in TrypLE for about 3-6 minutes and triturated until cells become fully dissociated. Cell suspension was then filtered with about 40 μm mesh size strainer, before plated to planar at about 33% confluency (9.5×10⁴ cells/cm²). For OTT cultures, about 5×10⁵ cells were seeded in a 24-w plate Transwells insert (Corning, 3470). Different sizes of Transwells insert can be used as well.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Efficient Differentiation of Human iPSC-derived Pancreatic Ductal Cells for Planar, Organoid and Tissue Chip Applications

### Ethics Statement

Human cell lines and tissues were obtained or created at Cedars-Sinai under the auspices of the Cedars-Sinai Medical Center Institutional Review Board (IRB) approved protocols. Specifically, the iPSC cell lines and differentiation protocols in the present study were carried out in accordance with the guidelines approved by Stem Cell Research Oversight committee (SCRO) and IRB, under the auspices of IRB-SCRO Protocols Pro00032834 (iPSC Core Repository and Stem Cell Program) and Pro00036896 (Sareen Stem Cell Program). In vitro studies using human cell lines were conducted from participants that provided written informed consent for research studies. Remaining studies were conducted with post-mortem human specimens with appropriate IRB approvals.

### Induced Pluripotent Stem Cell Maintenance

CS07iCTR, CS03iCTR and EDi028-A iPSC lines were reprogrammed by the iPSC Core at Cedars- Sinai Medical Center from peripheral blood mononuclear cells obtained from 60-, 34- and 79-year-old male individuals. Extensive characterization was performed including pluripotency assays with flow cytometry, qRT-PCR and immunocytochemistry, G-band karyotyping, scorecard assay to assess differentiation potential, and confirmation of the absence of episomal plasmid genes. Cells were maintained and expanded at a 1:6 ratio using passaging reagent ReLeSR and cultured in mTeSR Plus Medium (both from Stem Cell Technologies, Vancouver, British Columbia, Canada) on Matrigel (Corning) coated plates.

### Pancreatic Progenitor Differentiation

iPSCs were single-cell dissociated using TrypLE (Gibco, 12604013), neutralized with base media, washed, resuspended after cell count, and plated onto Matrigel-coated plates at a density of 3.1×10⁵ cells/cm² using mTeSR+ and 10 μM Rho kinase Inhibitor (STEMCELL). The following day, cells were directed into Definitive Endoderm (DE) using Phase I medium, composed of base medium MCDB 131 (Fisher Sci) supplemented with 100 ng/ml Activin A (R&D), 2 μM CHIR99021 (Reprocell), and 10 μM Rho kinase Inhibitor (Cayman) for 1 day. For the next two days, the same base medium was used, but supplemented instead with 100 ng/mL Activin A and 5 ng/mL FGF2 (Peprotech). Following this phase, cells were directed to form Posterior Foregut (PFG) using Phase II medium, which was composed of the same base medium as Phase I but supplemented with 50 ng/mL FGF10 (Peprotech), 0.25 μM CHIR99021 and 50 ng/ml Noggin (Peprotech) for 2 days. To reach a Pancreatic Progenitor (PP) stage, cells were fed with Phase III medium, composed of DMEM base (Gibco), 1×Pen-Strep, 1×B27 without Vitamin A (Thermo Fisher), and Vitamin C (250 μM), supplemented with small compounds 50 ng/mL Noggin (Peprotech), 50 ng/mL FGF10 (Peprotech), 2 μM Retinoic Acid (Sigma), and 0.25 μM SANT1 (Sigma) for 2 days.

iPan Ductal Specification

After 1 day of Phase III, on Day 7, cells were single cell dissociated with TrypLE for 3-6 minutes, washed and seeded at 33% confluency (9.5×10⁴ cells/cm²) in Phase III media plus 10 μM Rho kinase Inhibitor. On Day 8 onwards, cells were treated with Phase IV media—addition of FGF10 (25 ng/ml), EGF (50 ng/ml), and sDLL-1 (25 ng/ml, Peprotech). The base media for phase III and IV consists of DMEM, lx Pen-Strep, 1×B27 without Vitamin A (Thermo Fisher), and Vitamin C (250 μM).

### Pancreatic Endocrine Specification

Cells were fed with the same Phase III media stated above for 4 days instead of the shortened version of 2 days for iPan Ductal differentiation. Phase IV for Pancreatic Endocrine consists of the same base media, but supplemented with a different combination of small compounds—Noggin (50 ng/ml), EGF (100 ng/ml, PeproTech) and Nicotinamide (10 mM, Sigma) for 4 days.

iPan Ductal Organoids

Day 7 cells were single cell dissociated with TrypLE for 3-6 minutes, washed and resuspended in Matrigel at a density of 2.85 million cells/mL. 10 μL of cell suspension was seeded in each well of a 96-well U-bottom plate, or 40 μL seeded in each well of a 12-well plate, then incubated at 37° C. with the plate flipped upside-down for 30 min. Once the Matrigel dome is solidified, Phase III medium with Rho kinase Inhibitor was added for 24 h, then the medium was switched to Phase IV media and cultured for 2 weeks or longer, whilst feeding every 2 days.

iPan Ductal on Tissue-Chips

Prior to seeding, emulate chips were plasma activated and Matrigel coated. The following day, Day 7 cells were dissociated and reseeded into the top channel at a density of 10 million cells/mL in Phase III media plus 10 μM Rho kinase Inhibitor. The bottom channel was filled with only Phase III media plus 10 μM Rho kinase Inhibitor. 24 hours later, media was replaced with Phase IV media. For Chips in dynamic system, Chips were placed in ZOE and a flow rate of 30 μl/hr was initiated 48 hours post-seeding. Feeding was done every other day for the next 2 weeks.

### Organoids Seeded to Transwells and Planar

Organoids were incubated in Cell Recovery Solution (Corning) for an hour in 4° to remove Matrigel, then washed 3× with PBS. Organoids were then single cell dissociated in TrypLE for 3-6 minutes and triturated until cells become fully dissociated. Cell suspension was then filtered with 40 μm mesh size strainer, before plated to planar at 33% confluency (9.5×10⁴ cells/cm²). For OTT cultures, 5×10⁵ cells were seeded in a 24-w plate Transwells insert (Corning, 3470).

### Cryopreservation

Day 7 planar cells or Day 21+ organoids were single cell dissociated with TryPLE for 3-6 minutes, washed then resuspended in 50% CryoStor (Stem Cell Technologies) and 50% Phase III/IV base media, with 2×10⁶ cells per vial. When thawed, cells were washed and resuspended in culture media supplemented with 10 μM Rho kinase Inhibitor. Cells could be plated on planar at 33% confluency (9.5×10⁴ cells/cm²), resuspended in Matrigel at 2.85×10⁶ cells/mL for organoids, seeded on chips at 1×10⁷ million cells/mL or 5×10⁵ cells could be seeded on 24-w Transwells.

RNA extraction and qPCR

For organoids, Cell Recovery Solution (Corning) was added for an hour in 4° to remove Matrigel then washed 3× with PBS. Total RNA was isolated with RLT buffer (Qiagen), syringed with 23 G needle, then extracted with RNeasy Micro Kit (Qiagen) according to the manufacturer's instructions. The concentration and the purity of RNA was determined with NanoDrop. 1-2 μg RNA was reverse transcribed to cDNA High-Capacity cDNA Reverse Transcription kit (Thermo Fisher). Real-time qPCR was performed (technical replicates, n=3-4) using SYBR Master Mix (Applied Biosystems, #4472952) and specific primer sequences to each gene (Table 1), on a CFX384 Real Time system (Bio-Rad). Human RPL13 was used as the reference gene and relative expression was determined using 2-ΔΔ Ct method.

### Cryosectioning

Organoids were incubated in Cell Recovery Solution (Corning) for an hour in 4°. Organoid solution was transferred to 15-ml conical tube and centrifuged at 300 rpm for 1 minute. Pellet was resuspended in PBS and centrifuged again. Then organoids were fixed in 4% PFA for 15 minutes, washed with PBS then stored in 30% sucrose solution until organoids sank. Organoids were then embedded in OCT and sectioned at 10 μM on a cryostat. Sections were mounted onto slides and left to dry overnight.

### Immunofluorescence

Planar cultures were fixed with 4% PFA for 15 minutes, washed with PBS and then blocked overnight at 4° C. with 10% donkey serum containing 0.1% Triton-X100. After overnight incubation with primary antibodies at 4° C., cells were washed with 0.1% TWEEN-20 3× for 5 min, incubated with secondary antibodies for an hour at RT, and finally incubated with Hoechst 33342 (1:2000 dilution) for 15 minutes for nuclear counterstaining. Cryosectioned samples were similarly blocked and stained, but instead of adding Hoechst, ProLong™ Gold Antifade Mountant with DAPI (Fisher Scientific, P36931) was added onto the sections before sealing with a cover slip.

For whole mount organoid staining without removing the Matrigel, organoids were fixed with 4% PFA for 30 minutes at RT, washed with PBS 3× for 5 min on a shaker, and then permeabilized with 0.1% Saponin in PBS for 15 minutes at RT. After washing with PBS on a shaker, blocking buffer (10% NDS, 0.1% Triton X-100) was added overnight at 4° C. Organoids were then incubated overnight at 4° C. with primary antibodies, washed with PBS 3× for 5 min on shaker, and secondary antibodies added (1:200) for 2 hours at RT. After washing with PBS 3× for 5 min on a shaker, organoids were incubated in Cell Recovery Solution (Corning) for an hour in 4° C., resuspended in Matrigel and then seeded to an optical 96-well plate for imaging.

The following antibodies were used: SOX9 (1:250, Abcam AB5535), CK19 (1:100, Fisher Scientific AB7755), HNF1B (1:100, R&D AF3330-SP), CFTR (1:100, R&D MAB25031), eNac (1:100, R&D PA1920A), eCad (1:100, R&D AF648), ZO1 (1:50, Fisher Scientific 33-9100), OCT4 (1:250, Stemgent 09-0023), SSEA4 (1:100, Abcam ab16287), SOX17 (1:250, Fisher Scientific PAS-23382), FOXA2 (1:100, Abcam ab60721), NKX6.1 (1:25, DSHB F55A10), C-Peptide (1:25, DSHB GN-ID4), Donkey anti-Mouse IgG Alexa Fluor 488 (1:1000, Fisher Scientific A21206), Donkey anti-Mouse IgG Alexa Fluor 568 (1:1000, Fisher Scientific A11057), Donkey anti-Mouse IgG Alexa Fluor 647 (1:1000, Fisher Scientific A-31571).

### Imaging

Immunofluorescence images of planar cells were captured using ImageXpress Micro XLS (Molecular Devices). Meanwhile, immunofluorescence images of CHIPS, Transwells, sectioned and whole mount organoids were capture with Nikon Eclipse Ti Confocal microscope. Brightfield images of organoids were taken with EVOS XL Core (Fisher Scientific) then analyzed with ImageJ software. Image quantification was performed using CellProfiler Software (v3.1.9).

### Western Blot

Cells were lysed in RIPA buffer containing 50 mmol/L Tris (pH 7.4), 150 mmol/L NaCl, 1% deoxycholic acid, 1% Triton X-100, 0.1% SDS and a mix of protease and phosphatase inhibitors (Roche Applied Science, Basel, Switzerland). Protein extracts were resolved by SDS-PAGE for immunoblot analysis and membranes probed with primary and horseradish peroxidase (HRP)-conjugated secondary antibodies. Immunoreactive bands were visualized by chemiluminescence (ThermoFisher Scientific) and digital images captured using the PXi 6 Touch Imaging System (Syngene). To estimate protein levels, optical density values in each blot were expressed relative to those of the loading control (ERK, β-actin or GAPDH). The following antibodies were used: phospho-CREB (Ser133; #9198), CREB (48H2; # 9197), HMGB1 (# 6893), SOX9 (#82630) and corresponding HRP-linked secondary antibodies were from Cell Signaling Technology (Danvers, MA); CFTR antibody (#MAB25031) was purchased from R&D Systems, Inc (Minneapolis, MN), and CA2 (#sc-133111) from Santa Cruz Biotechnology (Dallas, TX). All primary antibodies were used at a 1:1000 dilution; secondary antibodies were used at a 1:10000 dilution.

### Iodide Efflux Assay

Assay was performed according to previous papers with some modifications. After removing culture media, planar cultures grown in 12- or 24-well plates were washed three times with 500 μL efflux buffer (136 mM NaNO3, 137 mM NaCl, 4.5 mM KH2PO4, 1 mM CaCl2, 1 mM MgCl2, 10 mM glucose, 5 mM HEPES, pH 7.2), and then loaded for 1 h at 37° C. with 600 μL NaI buffer (similar composition than the efflux buffer with NaI 136 mM replacing NaNO3). Extracellular NaI was then washed 4 times with 500 μL efflux buffer and cells incubated at 37° C. with 600 μL efflux buffer for 5 min to establish a stable baseline. Then, cells were left untreated or treated with 10 μM Forskolin (FSK) to promote CFTR-induced iodide efflux, and aliquots of media (60 μL each) collected at time 0 and at 1 min intervals for 8-10 min. For iPan Ductal on chips, supernatant was collected after 3 minutes of 136 mM NaNO3 then after 8 minutes of 136 mM NaNO3 containing 10 μM FSK. Iodide concentration was calculated using a Thermo Orion ion plus Iodide combination Electrode (#9653BNWP; Thermo Scientific) filled with iodide-sensitive electrolyte solution (#900063; Thermo Scientific). The electrode was immersed in 5-10 mL of 100 mM NaNO3 and voltage change measured by adding each aliquot serially. Standard curves were obtained using 10 μM, 100 μM, 1 mM Nal and 10 mM Nal solutions.

### Carbonic Anhydrase Assay

Pellets were lysed with buffer consisting of 150 mM NaCl, 10 mM Tris, 1% Triton X-100, pH=7.2. Bradford assay was used to measure protein concentration. Carbonic Anhydrase Activity Assay Kit (Biovision, #K472) was performed on lysates according to the manufacturer's instructions. Values were then normalized to protein concentration.

### Statistical Analyses

Data are presented as mean ±standard error of the mean (SEM). Statistical significance between groups was determined by One-way ANOVA. P values<0.05 were considered statistically significant. Statistical analyses and graphs were generated using GraphPad Prism 7 for Windows Software (GraphPad Software).

TABLE 1
List of Primers

| | | SEQ | | SEQ |
|---|---|---|---|---|
| | | ID | | ID |
| Gene | Forward 5′-3′ | NO: | Reverse 5′-3′ | NO: |
| PDX1 | CCAGTGGGCAGGCGG | 1 | AGGAACTCCTTCTCCAGCTC | 2 |
| | | | T | |
| GATA4 | CGTGTCCCAGACGTTCTCAG | 3 | CCTTGTGGGGAGAGCTTCAG | 4 |
| SOX9 | GCTCTGGAGACTTCTGAACG | 5 | CCGTTCTTCACCGACTTCCT | 6 |
| | A | | | |
| KRT19 | GTTCACCAGCCGGACTGAA | 7 | GCAGGTCAGTAACCTCGGAC | 8 |
| CA2 | TCAGGACAAAGCAGTGCTCA | 9 | TGTCCATCAAGTGAACCCCA | 10 |
| | A | | G | |
| HNF1B | GCACCCCTATGAAGACCCAG | 11 | GGACTGTCTGGTTGAATTGT | 12 |
| | | | CG | |
| FOXA2 | GGGAGCGGTGAAGATGGA | 13 | TCATGTTGCTCACGGAGGAG | 14 |
| | | | TA | |
| GATA6 | AGAAGCGCGTGCCTTCATC | 15 | TTTCTGCGCCATAAGGTGGT | 16 |
| SCNN1A | GTTTCACCAAGTGCCGGAAG | 17 | CCCATTCCTGGGATGTCACC | 18 |
| SCNN1B | GAACCTGCTCCCTCCCAGTC | 19 | GTGCATAGTGGCACCTGCT | 20 |
| SLC4A4 | GAACGCATCCGATTCATCTT | 21 | CTTGATCCACCTGGCTGTTT | 22 |
| SLC26A6 | ACTGCAAGATGATGCAGGTG | 23 | ATGAGGCTGTGGAAGTCTGG | 24 |
| CFTR | TGACCTTCTGCCTCTTACCA | 25 | CACTATCACTGGCACTGTTG | 26 |
| | | | C | |
| RLP13 | GGCTAAACAGGTACTGCTGG | 27 | AGGAAAGCCAGGTACTTCAA | 28 |
| | G | | CTT | |

TABLE 2
Base Media Formulation

| | | |
|---|---|---|
| Component | Company | Final |

Phase I-II Base

| MCDB 131 | Fisher Scientific | | |
|---|---|---|---|
| Glutamax | Fisher Scientific | 2 | mM |
| Pen/Strep | Sigma Aldrich | 1% | |
| Vitamin C | Sigma Aldrich | 250 | μM |
| BSA | VWR | 0.50% | |
| Sodium Bicarbonate | Sigma Aldrich | 1.5 | g/L |

Phase III-IV Base

| DMEM | Fisher Scientific | | |
|---|---|---|---|
| Pen/Strep | Sigma Aldrich | 1% | |
| B27 w/out Vitamin A | Fisher Scientific | 1% | |
| Vitamin C | Sigma Aldrich | 250 | μM |

TABLE 3
iPan Ductal Specification Media Formulation

| | | Base | | | | |
|---|---|---|---|---|---|---|
| Phase | Day | Media | Factors | | | |
| I | 1 | Phase | Activin A | 338-AC-05 | 100 | ng/ml |
| | | I-II | CHIR99021 | 040004 | 2 | μM |
| | | | Rhok Inh | 10005583 | 10 | μM |
| | 2 to 3 | Phase | Activin A | 338-AC-05 | 100 | ng/ml |
| | | I-II | Basic FGF | 100-18B | 5 | ng/ml |
| | | | (FGF2) | | | |
| II | 1 to 2 | Phase | FGF 10 | 100-26 | 50 | ng/ml |
| | | I-II | CHIR99021 | 040004 | 0.25 | μM |
| | | | Noggin | 120-10C | 50 | ng/ml |
| III | 1 to 2 | Phase | FGF 10 | 100-26 | 50 | ng/ml |
| | | III-IV | RA | R2625-100MG | 2 | μM |
| | | | Noggin | 120-10C | 50 | ng/ml |
| | | | SANT1 | S4572-5MG | 0.25 | μM |
| IV | 1 to 21+ | Phase | EGF | AF-100-15 | 50 | ng/ml |
| | | III-IV | FGF 10 | 100-26 | 25 | ng/ml |
| | | | sDLL-1 | 140-08 | 25 | ng/ml |

### Results

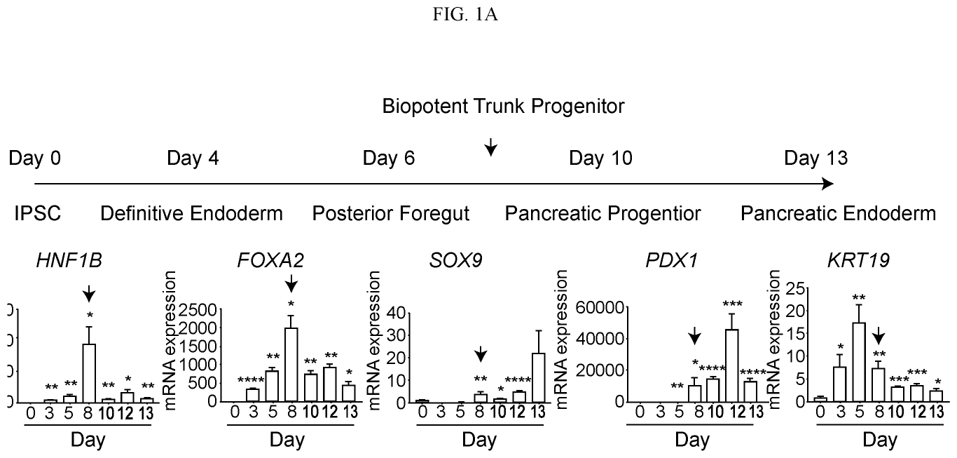

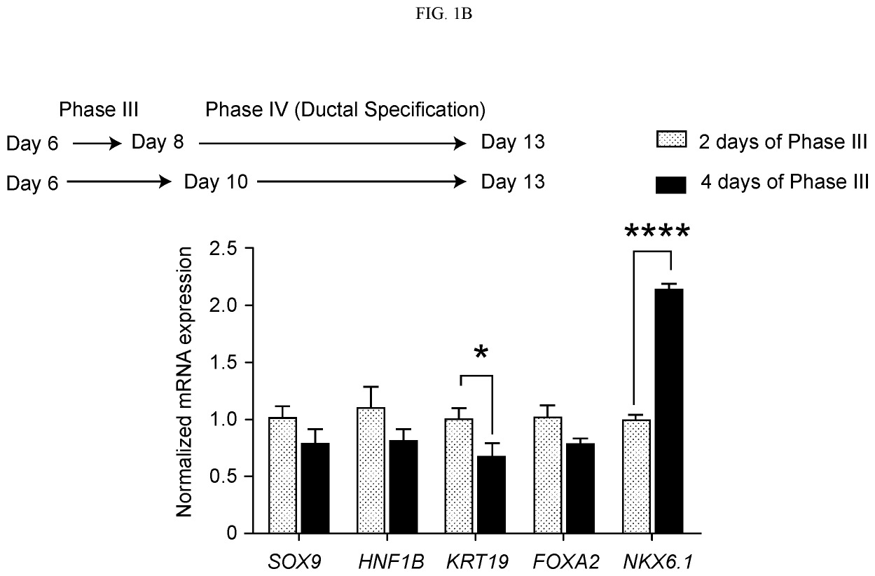

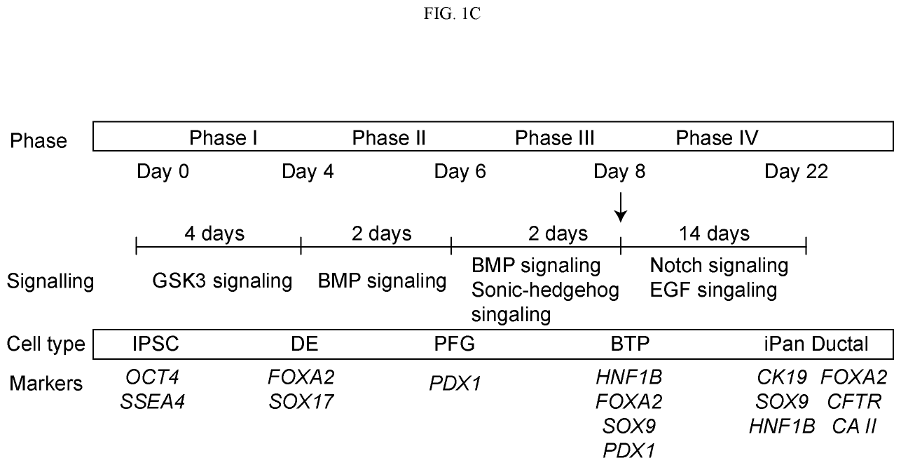

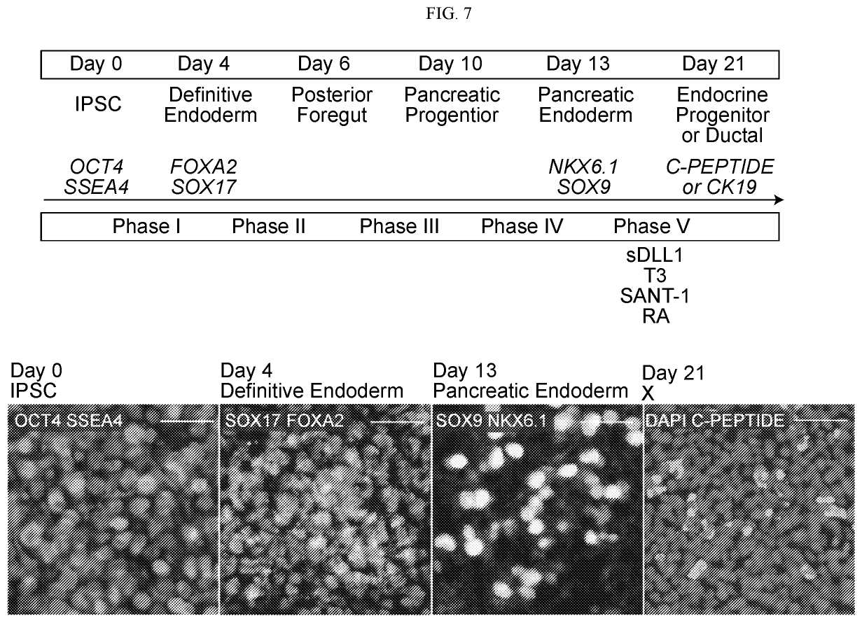

Notch signaling directs bipotent trunk progenitors toward a ductal lineage Pancreatic ductal and endocrine cells develop from a pool of bipotent trunk progenitors (BTP), and the bifurcation towards ductal lineage is mediated by Notch signaling. We found that 8 days of differentiation from iPSCs to be the best time to modulate Notch pathway as cells express BTP genes SOX9, HNF1B, KRT19 and FOXA2 at this time (FIG. 1A). A shortened treatment of Phase III media supplemented with FGF10, RA, SANT-1 and Noggin over 2 days, instead of 4 days as described in Memon et al., promoted increased expression levels of ductal markers and decreased levels of pancreatic endocrine marker, NKX6.1 (FIG. 1B). Earlier attempts at iPan Ductal consisted of 4 days of Phase III, then 4 days of pancreatic endoderm specification media followed by 2 weeks in the presence of the Notch-activating ligand, soluble Delta Like Canonical Notch Ligand 1 (sDLL1). Human sDLL-1 comprises the extracellular signaling domain of DLL1 that is a member of a family of single-pass type I trans-membrane proteins that serve as ligands for Notch receptors. Unfortunately, pancreatic endocrine marker C-Peptide was elevated, and no increase in ductal markers were found on Day 21 (FIG. 7), indicating that the initiating ductal specification at the pancreatic endoderm stage was too late to effectively elicit ductal phenotype by activation of Notch signaling.

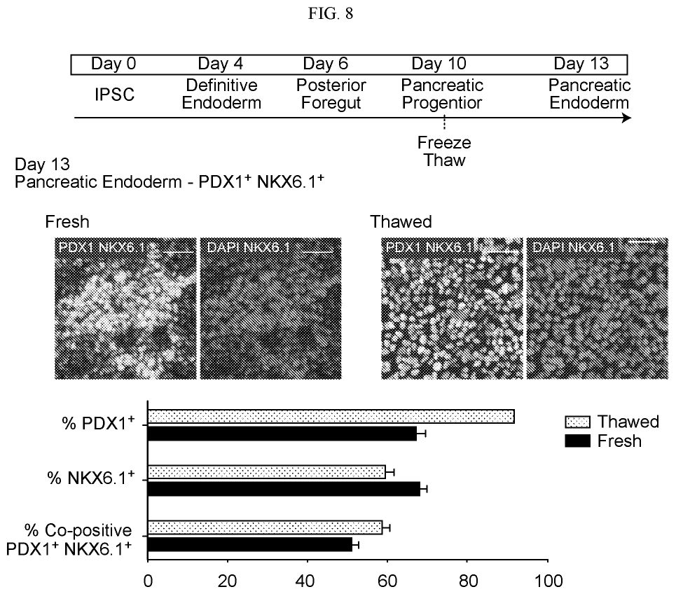

iPSCs differentiated stepwise to definitive endoderm, then posterior foregut can subsequently be directed to pancreatic progenitors (FIG. 1A). An established pancreatic endocrine differentiation protocol was starting point for deriving our iPan Ductal protocol. Since our laboratory has reproducibly generated PDX1⁺ SOX9⁺ pancreatic progenitors and SOX9⁺ NKX6.1⁺ pancreatic endoderm cells with multiple cell lines (FIG. 8), the next goal was to reverse engineer the protocol to determine the bipotent trunk phase, at which point we could then promote ductal differentiation. FIG. 1A depicts a timeline of mRNA expression during pancreatic endoderm differentiation. Data from Day 8 of differentiation show emergence of a bipotent trunk cell population expressing high levels of HFN1B, FOXA2, SOX9 and PDX1 genes followed by a decline along the endocrine differentiation axis (FIG. 1A). These results imply that Day 8 is an opportune time point for inducing Notch signaling to direct differentiation towards ductal cells. Determining the temporal window of modulating Notch is critical as mistiming the sDLL1 treatment later produced a higher amount of C-Peptide⁺ endocrine cells (FIG. 7).

To confirm that Phase III should be shortened, we tested ductal differentiation protocol with either 2 or 4 days of Phase III treatment, followed by ductal specification in Phase IV, which entailed addition of EGF, FGF10 and sDLL1 to the cultures. After 2 days of Phase III treatment, we found increased levels of ductal markers, whereas a 4-day Phase III treatment was associated with increased levels of the pancreatic endocrine NKX6.1 (FIG. 1B). FIG. 1C shows the final version of our optimized iPan Ductal differentiation schedule with 4 days of Definitive Endoderm specification, 2 days of Posterior Foregut specification, 2 days of Bipotent Trunk Progenitors specification, then terminal iPan Ductal maturation for 2 weeks or longer.

Cryopreserved iPSC-Derived Pancreatic Progenitor Cells Provide Versatility in Different Applications

Optimization of these types of experiments is slow and arduous as expansion, differentiation and cellular characterization of each iPSC line typically requires about five weeks to accomplish. Therefore, we established a more efficient and scalable method that allowed us to differentiate and cryopreserve large batches of pancreatic progenitors with less batch-to-batch differentiation variability. We have confirmed that thawed and fresh cells have equal differentiation efficiency. FIG. 8 shows a comparison of marker expression and quantification of fresh and thawed cells, with 51% and 59% of co-positive PDX1⁺ NKX6.1³⁰ cells, respectively.

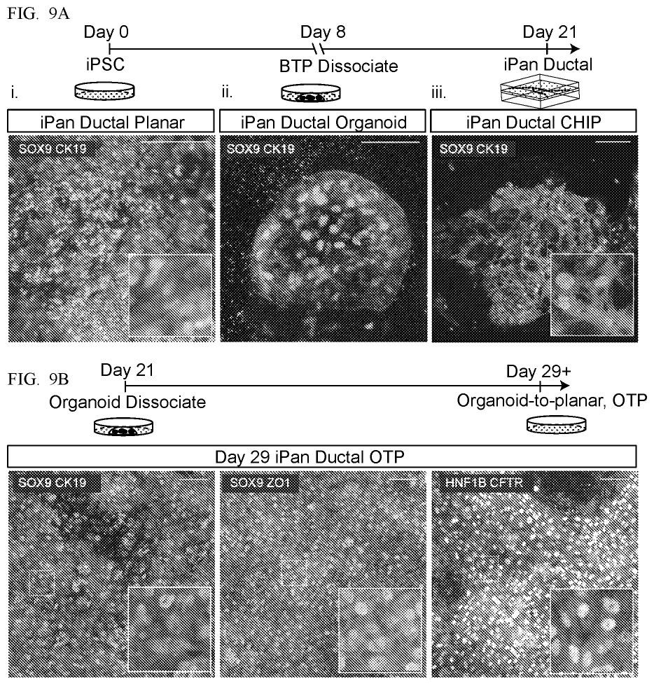

The cryopreserved pancreatic progenitors also thawed and then differentiated to iPan Ductal planar, organoids and Tissue-chip formats show reliable expression of SOX9³⁰ CK19⁺ cells (FIG. 9). The ability to freeze progenitors allowed us to run a quality check on the differentiation efficiency on a small subset of cells before performing functional assays. Batches of cells frozen at the pancreatic progenitor stage that fail to exhibit pancreatic ductal markers, SOX9 and CK19, when analyzed at Day 21, would not be thawed to produce organoids and planar cultures for swelling or iodide efflux assays. This testing regimen potentially saved us 3 weeks of differentiation, media, and reagents each time it was followed.

iPan Ductal Maturation Requires Cells to be Passaged and Cultured in Presence of EGF Without Noggin and Nicotinamide

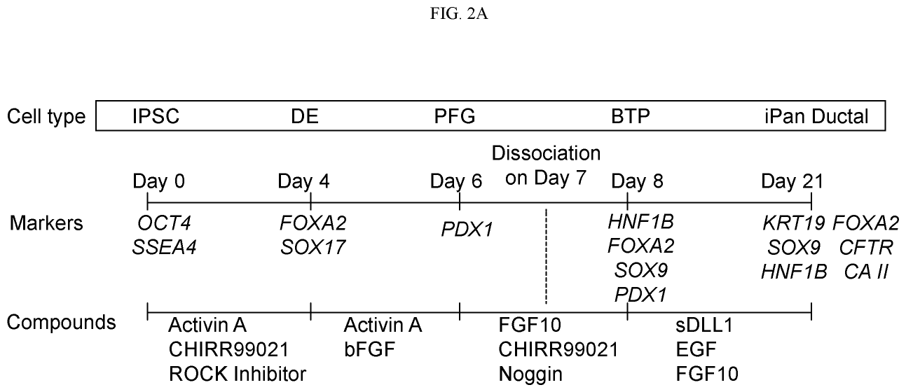

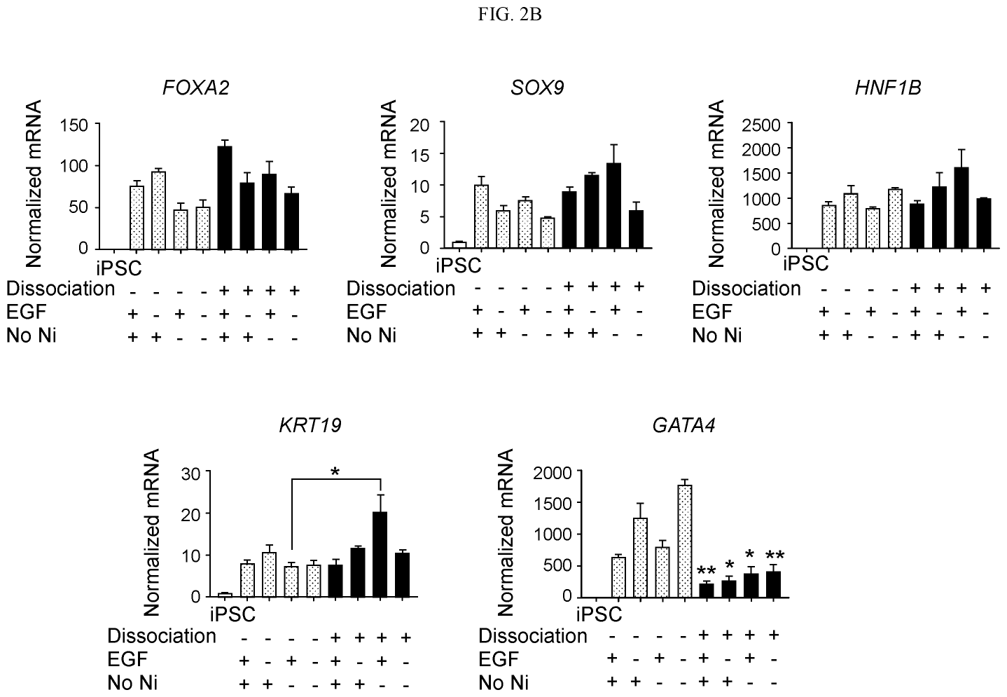

To optimize the ductal maturation phase, the effect of dissociating pancreatic progenitors on Day 7 and adding growth factor EGF and the Notch activating ligand sDLL-1 to the cultures was explored. FGF10 was also added at this time to increase proliferation of pancreatic cells. Based on our results, we incorporated a dissociation step prior to initiating EGF and Notch signaling in our defined iPan Ductal protocol (FIG. 2A). Dissociation of pancreatic progenitor cells inhibited acinar differentiation by Day 21 (FIG. 2B).

On Day 7, midway through Phase III, some cell cultures were dissociated into single cell suspension and reseeded. Dissociation was associated with a significant decrease in expression of the pancreatic progenitor and acinar marker GATA4 (FIG. 2B). Also, dissociated cells exposed to EGF responded with increased survival and confluency based on immunocytochemistry (not shown). Immunoblotting confirmed increased levels of CK19 and PDX1 proteins in all conditions relative to iPSCs (data not shown).

Noggin and Nicotinamide have been often included in pancreatic endocrine and exocrine differentiation protocols derived from hESCs. Conditions with Noggin and Nicotinamide (No Ni) were included to test whether these additives could promote further development towards a ductal fate without a population of endocrine specified NGN3⁺ cells arising. Since there were no apparent differences observed to result from the conditions containing Noggin and Nicotinamide, these 2 reagents were subsequently excluded. Conditions with EGF and without No Ni show a significant increase in KRT19 in the dissociated condition in comparison to that of the non-dissociated condition (FIG. 2B).

Bipotent Trunk progenitors thrive in multiple 2D and 3D cell culture formats

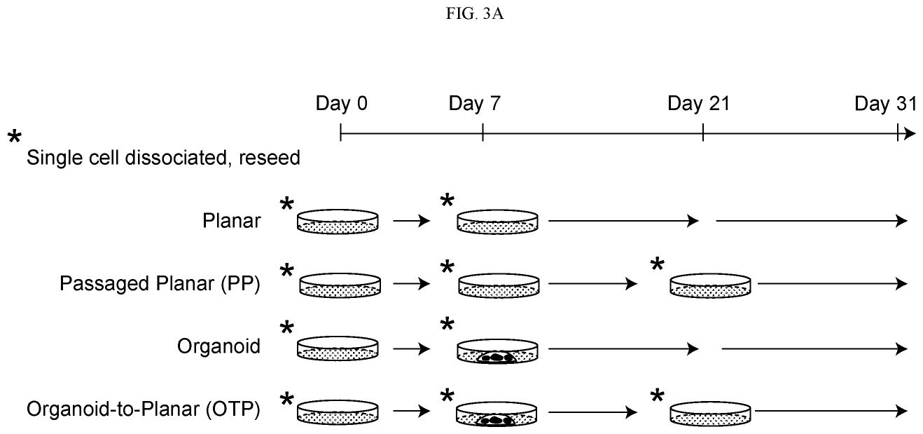

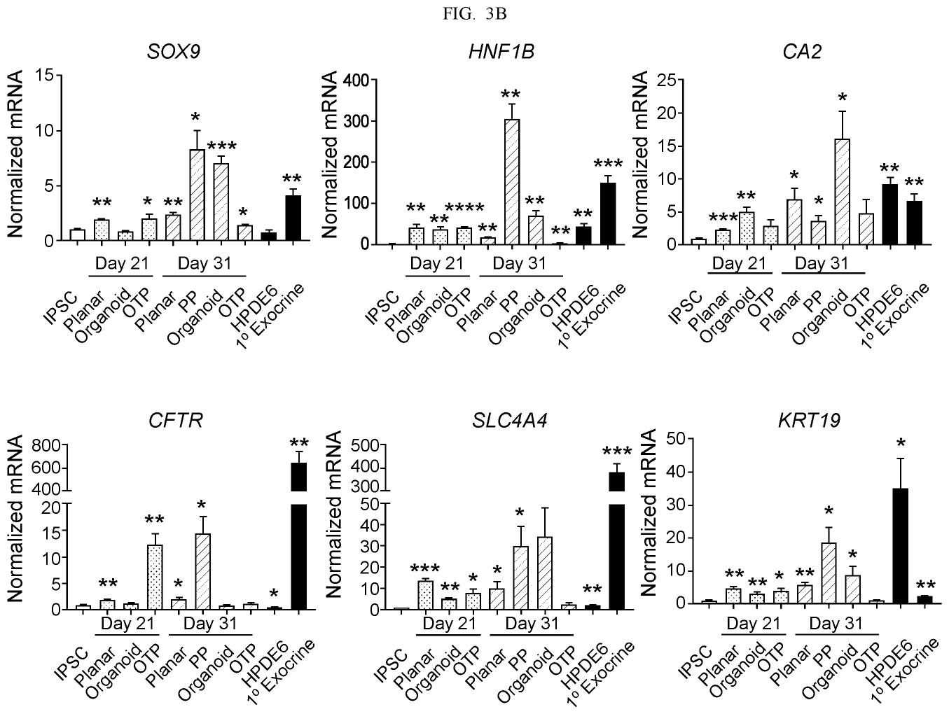

The ability to dissociate iPan Ductal cells without disrupting their viability and differentiation efficiency provides an additional ability to dissociate and reseed iPan Ductal into multiple 2D and 3D cell culture formats for an extended period. As depicted schematically in the diagram shown in FIG. 3A, planar cultures can be dissociated and reseeded on (or about) Day 21 to prevent detachment or over-confluence of cells, thereby extending their longevity. On the other hand, we successfully prepared iPan Ductal organoids by aggregating single cells into Matrigel domes (as described in methods section). We observed that our organoids can be cultured for longer than 2 weeks, or alternatively, they can be dissociated on Day 21 into a planar format, termed organoid-to-planar (OTP) cultures. This allows us to culture dissociated organoids in planar form for longer than 31 days, whereas planar cultures typically only persist up to Day 26-31 before detachment occurs. FIG. 9 shows immunocytochemistry of OTP cells confirming expression of ductal proteins SOX9, HNF1B, CK19 and CFTR; and a tight junction marker, Z01.

FIG. 3B shows that ductal genes are upregulated in all formats — planar, passaged planar (PP), OTP and organoids. Day 31 organoids exhibited increased ductal markers in comparison to Day 21 organoids, indicating that organoids continue to mature with time. In contrast, Day 21 OTP had higher ductal SOX9, CFTR, SLC4A4, KRT19 gene expression than that of Day 31 OTP. Day 31 planar cultures also did not show significant improvement over Day 21 planar. In fact, Day 31 OTP showed the lowest amount of ductal expression of all the formats and timepoints we examined, indicating that a peak level of ductal differentiation occurs in these cultures sometime before Day 31. On Day 31, passaged planar showed significantly higher ductal SOX9, HNF1B, CFTR, SLC4A4 and KRT19 gene expression than all formats and timepoints. These data suggest that, not only does passaging improve adherence and longevity of planar cells, but it also can improve the ductal gene expression in these cultures.

Planar iPan Ductal Cultures are Functionally Mature

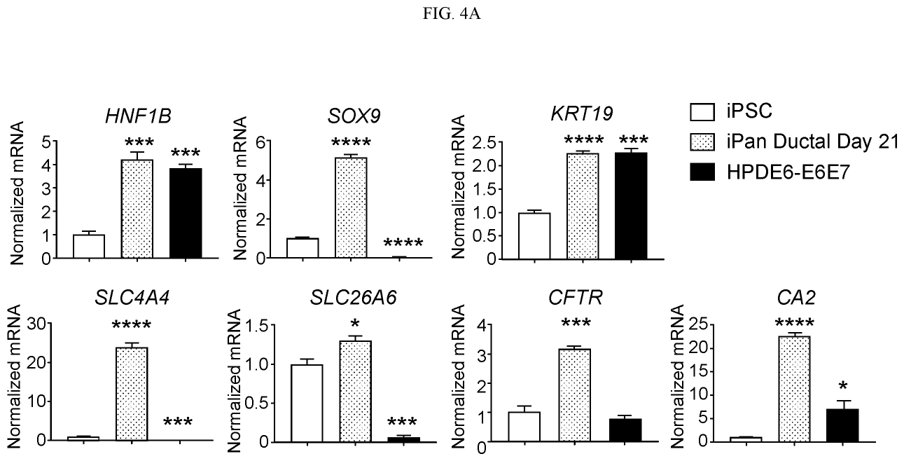

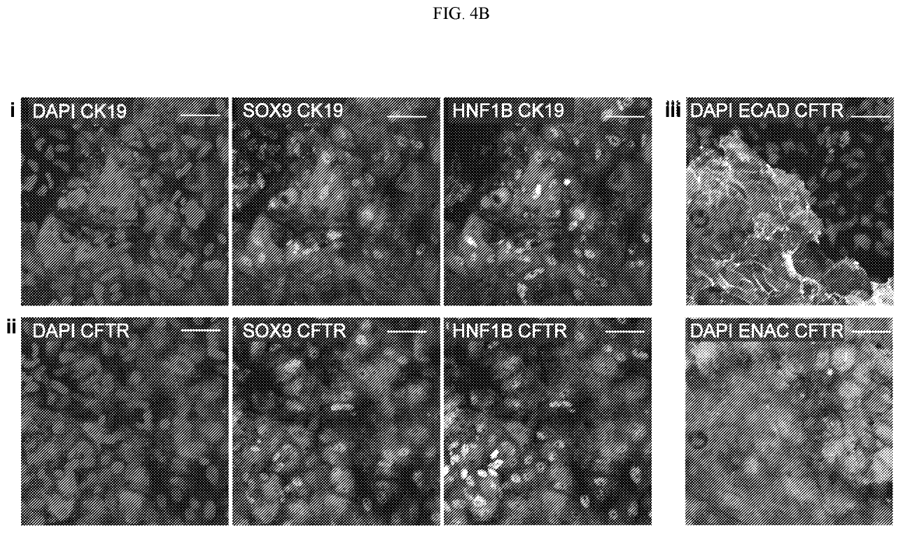

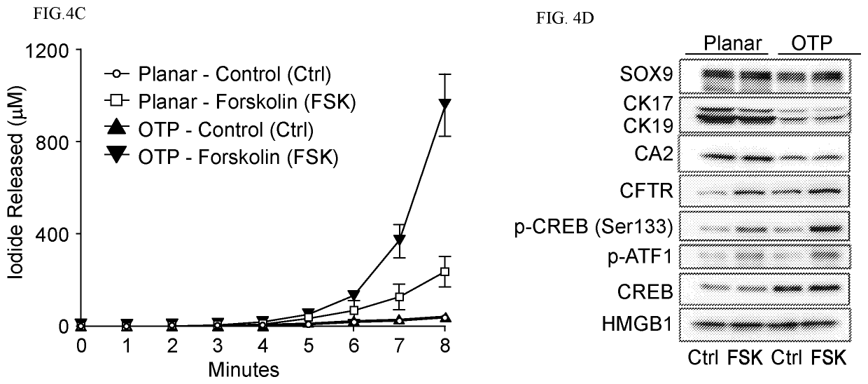

By Day 21, iPan Ductal cells showed upregulation of ductal transcription factors HNF1B, SOX9, cytoskeletal KRT19, transporters/channel proteins CFTR, SLC26A6, SLC4A4 and enzyme CA2 relative to iPSCs (FIG. 4A). Positive staining's of ductal proteins CK19, SOX9, HNF1B, CFTR, sodium transporter ENaC and adherent junctional protein E-Cadherin (CDH1) were also observed in these cultures (FIG. 4B).

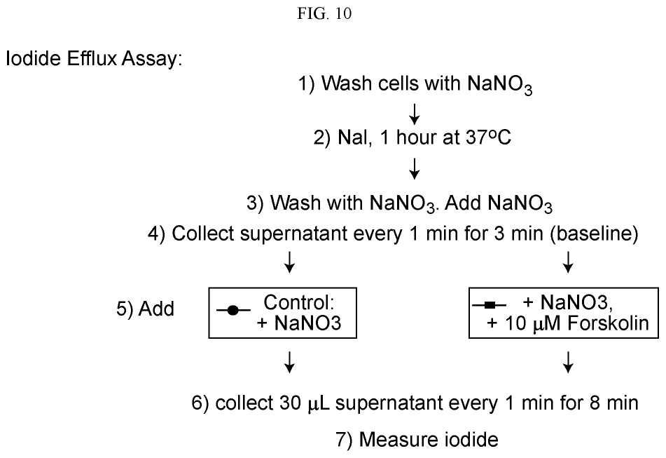

Iodide efflux assays were performed to measure the function of CFTR in these iPan Ductal cultures. Forskolin (FSK) is a cAMP-inducing agent that induces opening of the CFTR channels and allows the expulsion of chloride and iodide into the media. cAMP stimulates the protein kinase A-mediated phosphorylation of CREB at Ser133 and ATF-1 at Ser63. FSK was added to Day 21 iPan Ductal cells, the media was collected, and iodide levels were measured with an iodide electrode. FIG. 10 depicts the schematic workflow of the assay. FIG. 4C shows increased levels of iodide released into the media in condition with FSK, for both planar and OTP cultures, in comparison with the control condition containing NaNO3 buffer only.

Cells were lysed after 2 rounds of 15-min FSK treatments, introduced at 24 h prior to and just before collection. Lysates were probed for ductal markers (SOX9, CK19, CA2, CFTR) to confirm ductal identity and probed for phosphorylated CREB and ATF-1 associated with cAMP signaling pathway induced when FSK was introduced to open the CFTR channels. Our data indicated that both planar and OTP cultures exhibited CFTR activation, i.e., functional evidence of ductal identity and concomitant increases in p-CREB and p-ATF1 upon FSK stimulation (FIG. 4D). These data are consistent with PKA-mediated opening of the CFTR channel by its direct phosphorylation at regulatory sites such as Ser660. Interestingly, there also appears to be an increase in CFTR upon addition of FSK. Functional iPan Ductal organoids express CFTR and respond to forskolin

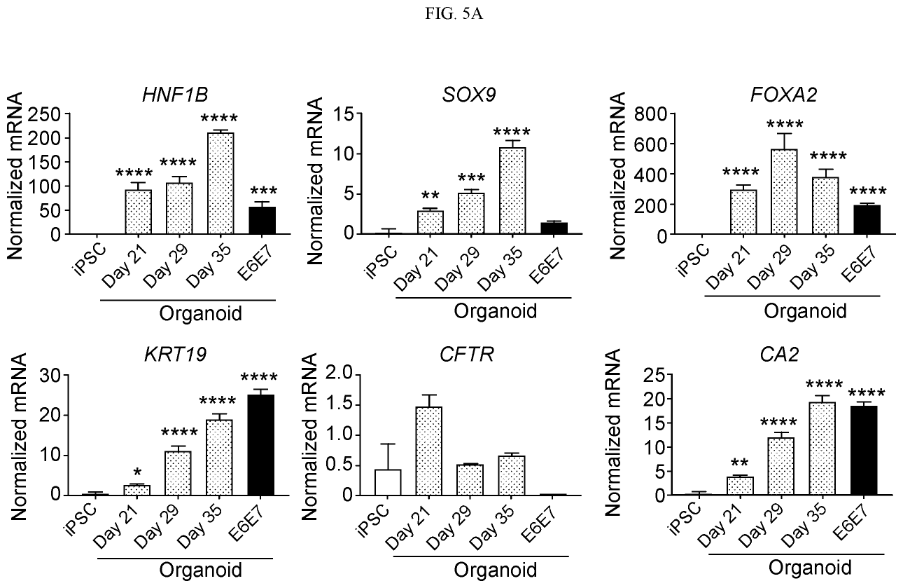

FIG. 5A shows a significant increase in RNA expression of HNF1B, SOX9, FOXA2, KRT19, and CA2 from iPSCs to Day 21-35 iPan Ductal organoids. Human Pancreatic Ductal Epithelium primary cell line, HPDE6-E6E7, were cultured to organoids and used as a positive control. There appears to be higher expression of ductal genes HNF1B, SOX9, KRT19, and CA2 on Day 35 organoids compared to Day 21 and 29. CFTR was not upregulated in either iPan Ductal organoids or E6E7 organoids, but CFTR protein expression was observed in FIG. 5Bi. Sectioned organoids show CDH1, EnaC, CFTR and CK19.

Whole mount organoids show SOX9 and CK19. Z=0 plane shows hollow lumen in the center and ductal cells on the periphery (FIG. 5Bii).

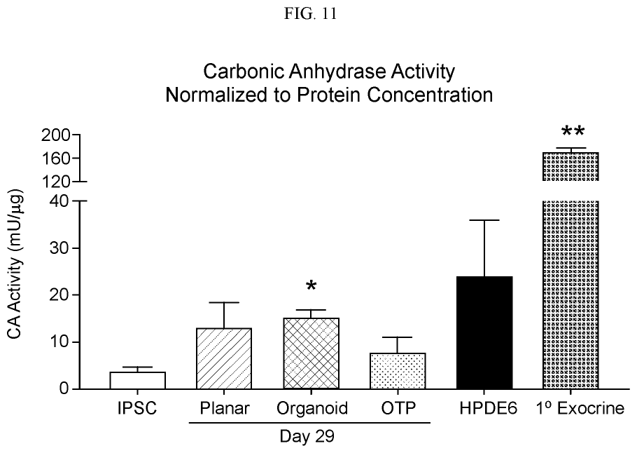

Since forskolin-induced swelling is known to be CFTR-dependent in different organoid-based systems by coupling ion transport to fluid transport, we assessed functional maturation of iPan Ductal organoids upon forskolin (FSK) treatment. FSK treated organoids show significant increase in fluid secretion and swelling compared to baseline of the organoids when not stimulated (FIG. 5C i and ii). Whereas expression levels of CFTR gene was not demonstrably high (FIG. 3, 5), the CFTR protein and function were measurable in organoids (FIG. 5). This data is supported by the observation that carbonic anhydrase 2 (CA2) which is a zinc-metalloenzyme catalyzing the reversible hydration of CO2 to maintain acid-base balance, pH regulation, and CO2 transport and critical to pancreatic ductal function, exhibited both increased CA2 gene expression and enzymatic activity in iPan Ductal organoids (FIG. 11).

iPan Ductal Cells are Functional After Adapting to Microfluidic Tissue-Chip and Transwell Systems

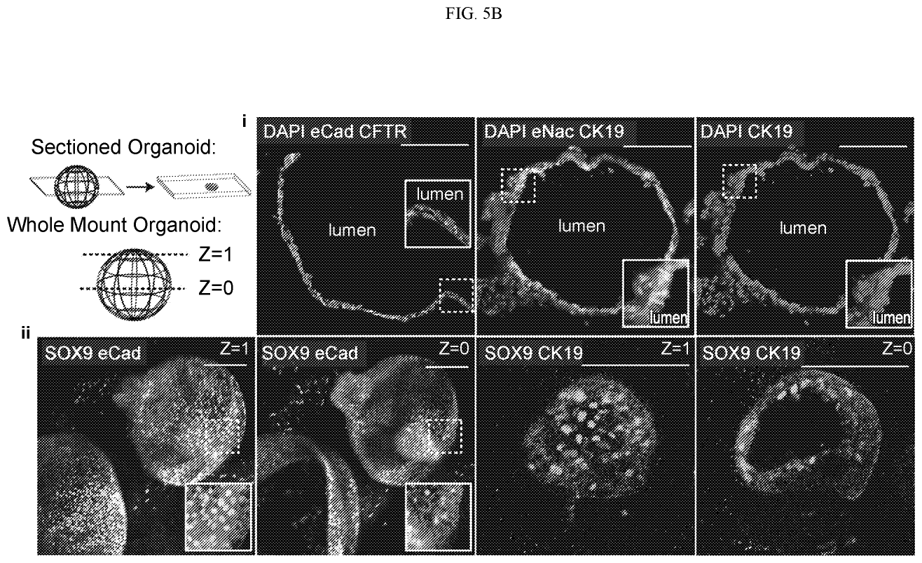

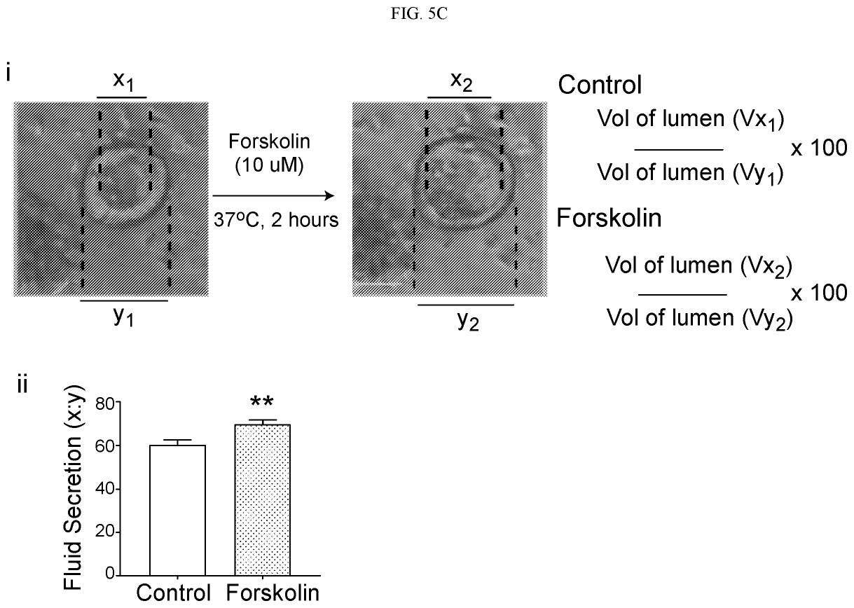

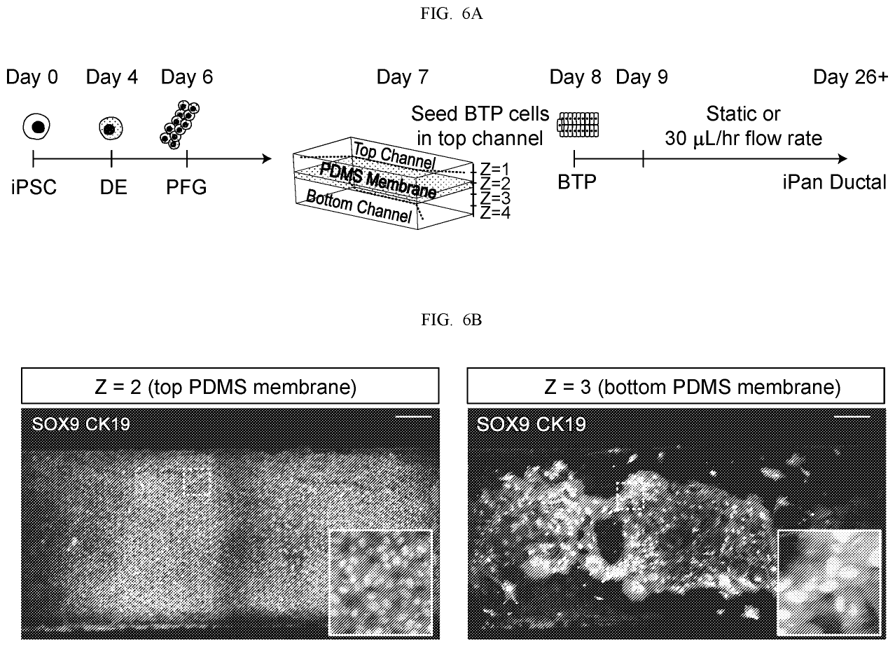

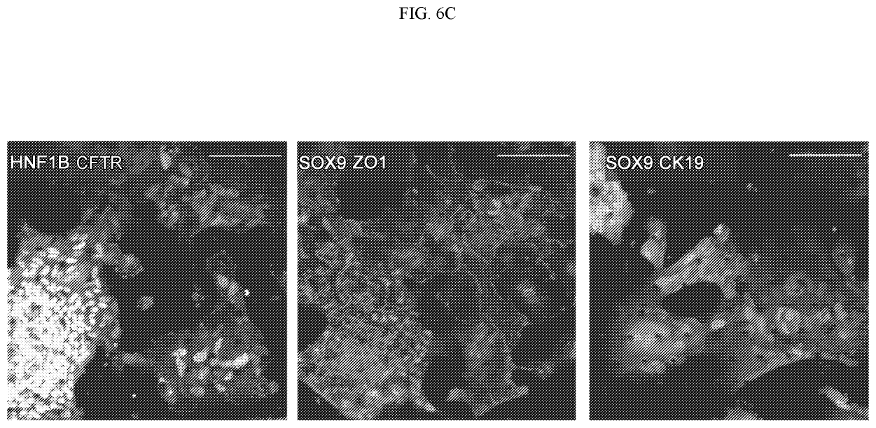

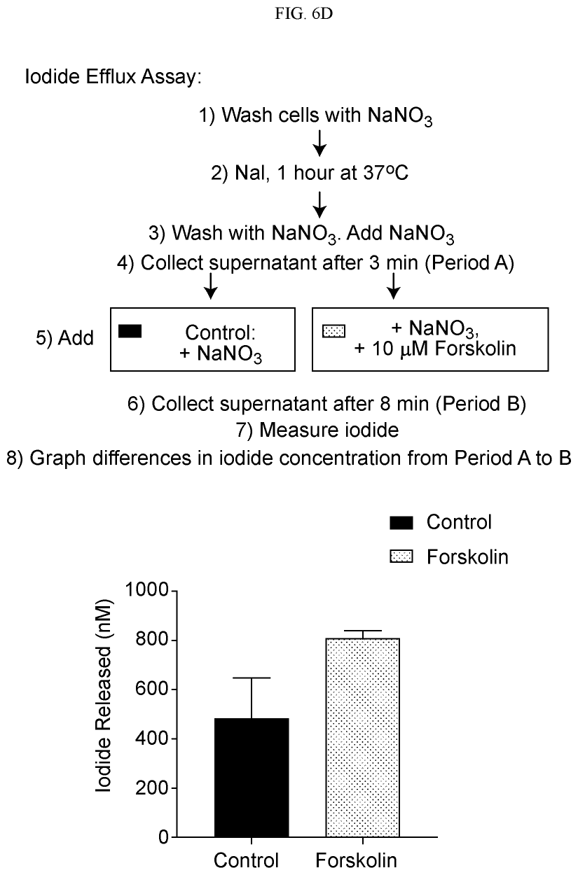

Seeding cells on microfluidic Tissue-chips and Transwell systems allows us to better emulate the dynamic flow of the in vivo environment and more closely approximate a polarized ductal epithelium in which CFTR normally localizes to the apical membrane. Therefore, we dissociated BTP cells and seeded them into the top channel of microfluidic chips. Dynamic flow was introduced 48 hours later by changing extracellular medium with a flow rate of 30 μl/hr (FIG. 6A). FIG. 6B shows Day 26 cells cultured in chips in medium without flow, while FIG. 6C shows Day 26 cells with 30 μL/hr flow rate. The cells cultured under both conditions exhibited expression of ductal proteins, SOX9 and CK19. Dynamic chip also shows HNF1B, CFTR and tight junction marker Z01. Iodide efflux assay was performed according to the workflow shown in FIG. 6D. Cells on chips were either exposed to FSK for 8 min, then supernatant was collected, and iodide levels measured. The graph (FIG. 6D) shows an increase in iodide released in the medium from the chip treated with FSK, as compared to the untreated control.

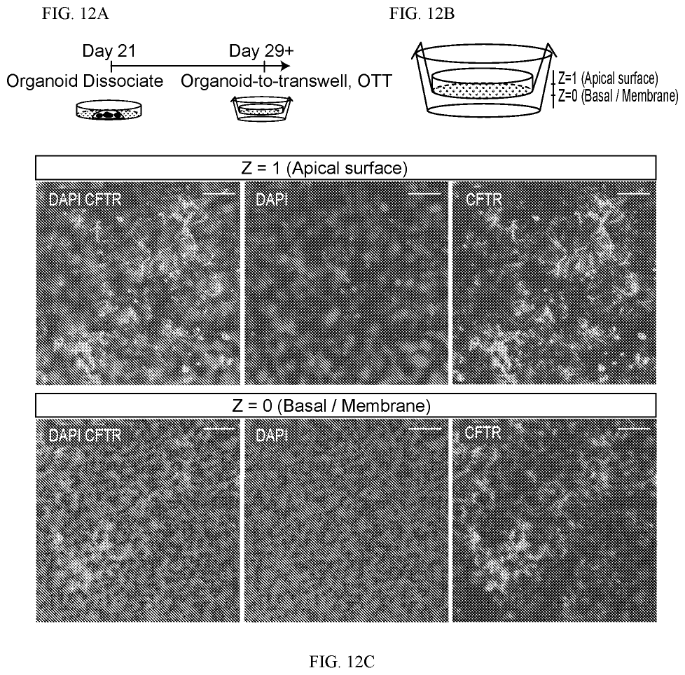

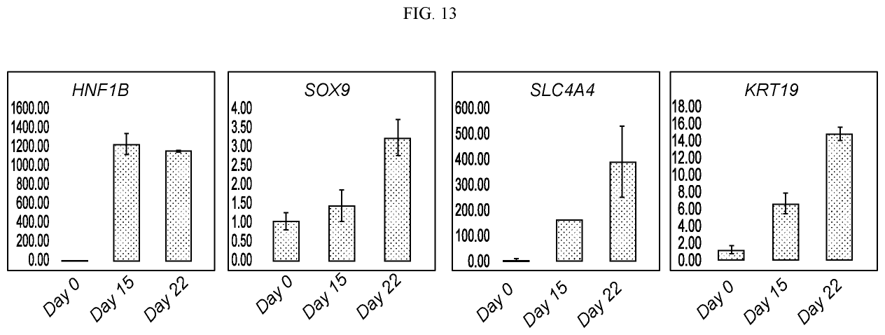

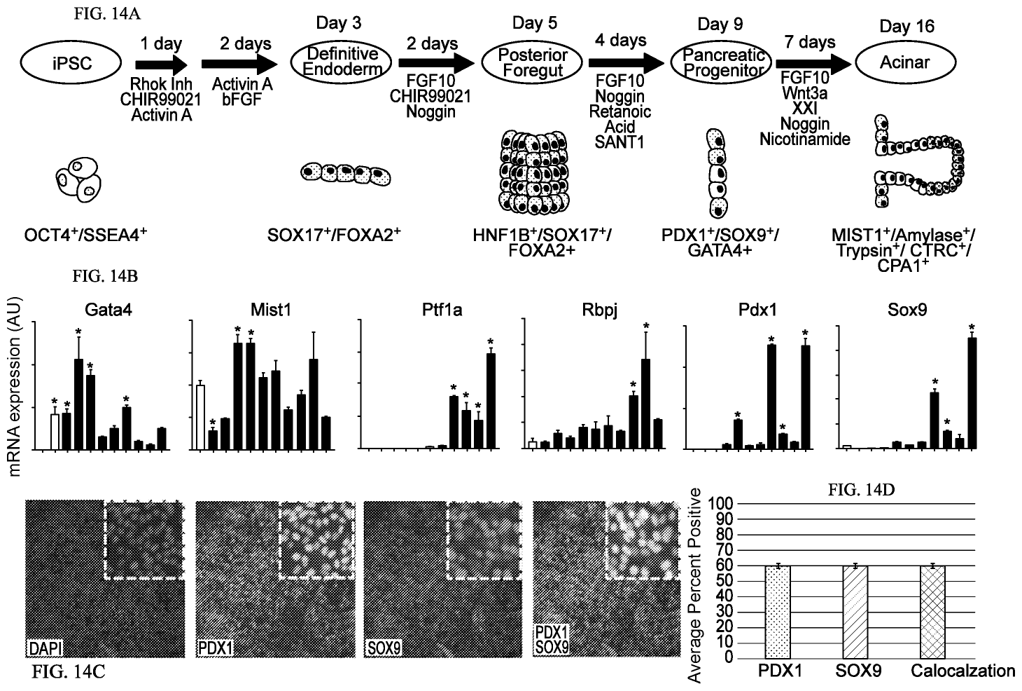

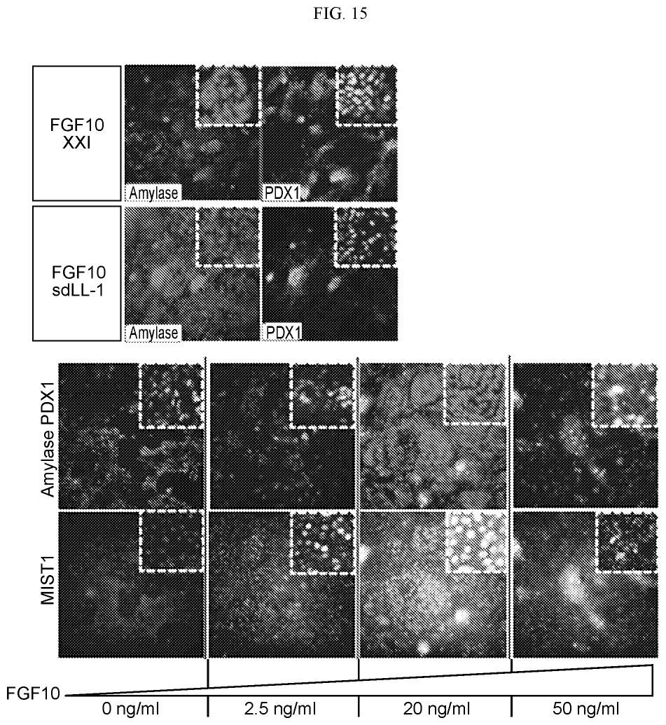

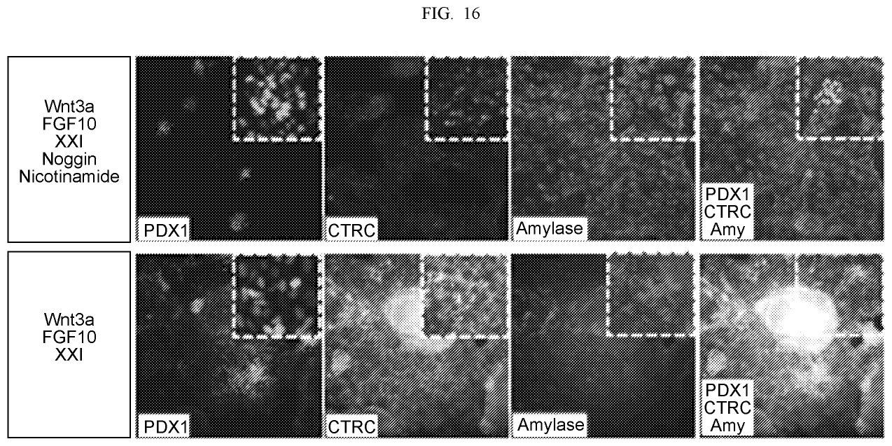

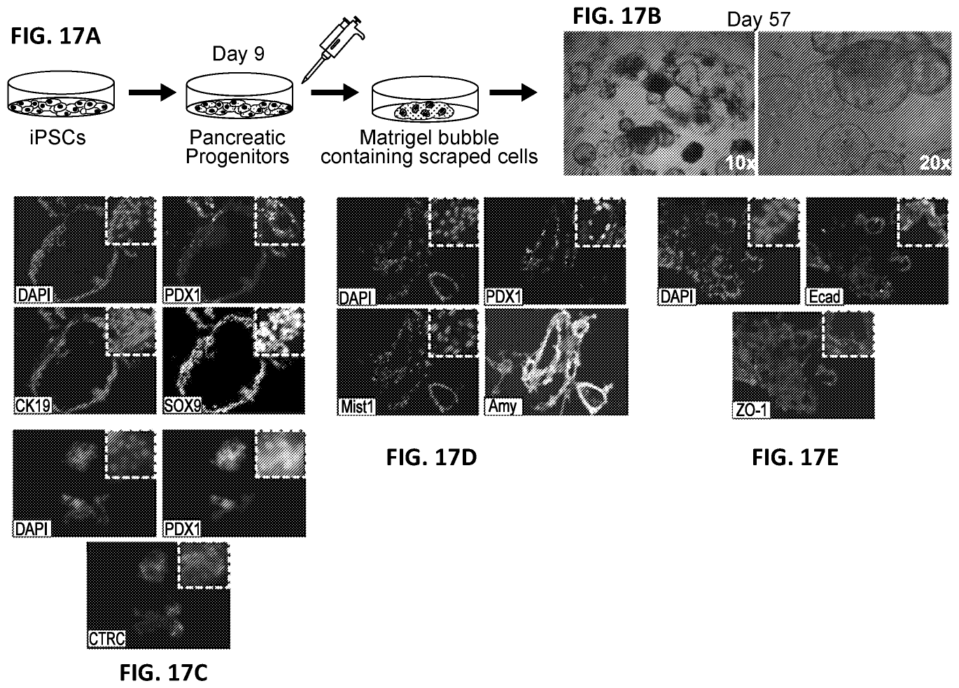

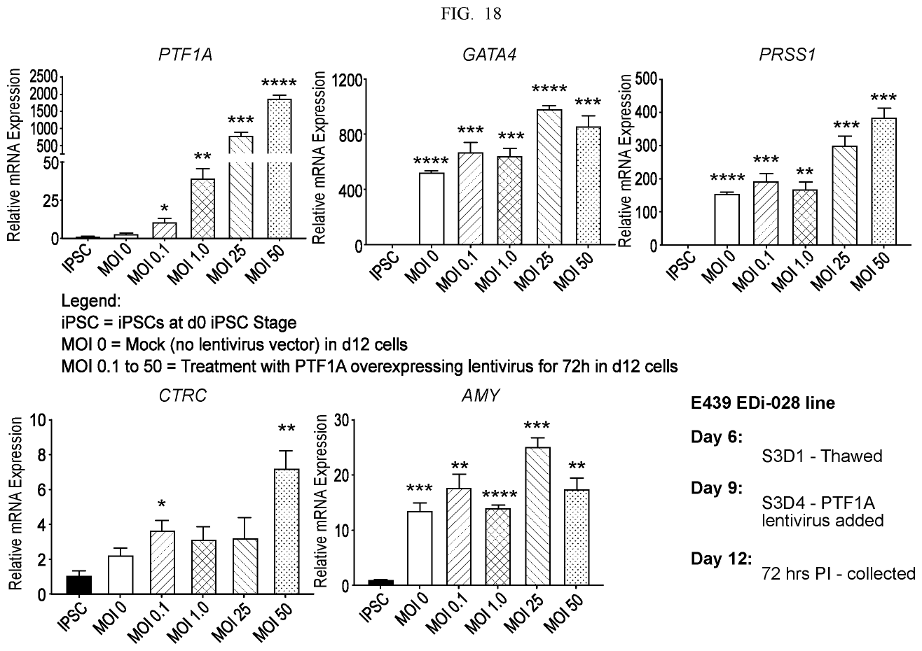

We next established a Transwell system by a process like that for OTP cultures. Day 21 organoids were dissociated and seeded on Transwells for up to Day 29 or longer (FIG. 12). FIG. 12 shows a cross-sectional image of the cells seeded on Transwell membranes where Z=0 refers to the basal aspect of the cells in contact with the Transwell membrane, while Z=1 indicates the apical surface of the ductal epithelium. Distinct CFTR protein expression is observed on the apical section of iPan Ductal cell culture in the Transwell system (FIG. 12).

### Example 2

### Reliable Generation of Functional Human iPSC-Derived Pancreatic Acinar Cells in Adherent and Organoid Cultures

Human iPSC Culture into Pancreatic Progenitors

All iPSC lines used were obtained from the iPSC Core at Cedars-Sinai. Cells were cultured on MATRIGEL (Corning Life Sciences) and fed with mTeSR Plus with mTeSR Plus supplement (Stem Cell Technologies) every other day. Passaging was done weekly and consisted of cells being dissociated with ReleaSR (Stem Cell Technologies) and then seeded at lower density with a split ratio of 1:5. To begin differentiation, cells were single-cell dissociated via accutase (Stem Cell Technologies) and then seeded onto MATRIGEL coated plates at 300,000 cells/cm^{2.} The day following seeding was considered “Day 1”. From this point, cells were fed daily. On Day 1, cells were fed with a base medium of MCDB 131 (Fisher Sci) supplemented with 2 mM Glutamax, 1% PSA, 250 uM Vitamin C, 0.50% BSA and 1.5g/L sodium bicarbonate. To this media, 100 ng/ml Activin A, 2 uM CHIR99021, and 10 uM Rho kinase Inhibitor was added. The following two days the cells were given the same base media but supplemented with 100 ng/mL Activin A and 5 ng/mL FGF2 to bring the cells to the definitive endoderm stage. This was followed with two more days of the same base media but supplemented with 50 ng/mL FGF10, 0.25 uM CHIR99021, and 50 ng/mL Noggin. At this time point of five days, cells had a posterior foregut identity.

The differentiation to pancreatic progenitors was performed with a different base medium of DMEM supplemented with 1% PSA, 1% B27 without Vitamin A, and 250uM Vitamin C. For four days, cells were given this DMEM base with 50 ng/mL Noggin, 50ng/mL FGF10, 2uM Retinoic Acid, and 0.25uM SANT1.

Differentiation of iPSC-derived acini

Using the same DMEM base media, cells were then treated daily for seven days with XXI, FGF10, Noggin, Nicotinamide, and Murine Wnt3a (Peprotech). By the end of this stage, cell clustering could be seen.

### Production of Pancreatic Organoids

For the first method of organoid culture favoring more acinar organoids, at the end of the acinar stage (day 16), cells were mechanically dissociated into clumps using a cell scraper followed by gentle trituration. Cells were seeded into Matrigel at a concentration of 1×6well/1040 uL Matrigel, and plated in 15 uL bubbles into 96 well round bottom plates. Cells were allowed to sit for 15 minutes at 37 C before being given 100 uL DMEM base media with FGF10, Noggin, Nicotinamide, and Murine Wnt3a every other day for 44 days.

For the second method of organoid culture, pancreatic progenitors (day 7) were single cell dissociated and resuspended in Matrigel at a density of 2 million cells/mL. 10 uL of cell suspension was seeded in each well of a 96 U-bottom plate then incubated at 37C for 15 minutes flipped. Then cells were fed with 100 ul of ductal media with FGF10, EGF and sDLL1 every 2-3 days for at least 2 weeks. The second method of organoid culture favor more ductal organoids Concentrations are: 25 ng/ml FGF10, about 50 ng/ml EGF, and about 50 ng/ml sDLL-1.

### Cryosectioning

Organoids were fixed for 15 minutes in 4% paraformaldehyde in PBS. Following this, they were washed and sunk in a 30% sucrose solution. Organoids were embedded in OCT before being sectioned in 10-14 um slices and placed onto slides.

### ICC

Planar fixed samples were fixed with 4% paraformaldehyde in PBS for 15 minutes. Following this, they were washed three times with PBS and then incubated with 10% donkey serum with 0.1% Triton-X for one hour. Primary antibodies diluted in the 10% blocking serum were added, and the samples were let incubate for three hours at room temperature or overnight at 4 C. From here, the samples were washed three times for five minutes each with 0.1% TWEEN in PBS. Secondary antibodies diluted at 1:1000 in the blocking serum were added and the samples were allowed to sit one hour at room temperature. Three rounds of five-minute washes with PBS were performed before Hoechst was added at a 1:2000 dilution in PBS. After a 15-minute room temperature incubation, samples were washed with PBS three times before being imaged.

For the cryosectioned samples mounted onto slides, a similar procedure was used, with the exception that the samples were washed three times for five minutes each in PBS before beginning blocking. The blocking serum was also a 5% donkey serum with 0.25% Triton-X. Each subsequent wash step occurred for 10 minutes. Instead of using Hoechst, ProLong™ Gold Antifade Mountant with DAPI (company) was used.

qPCR

RNA was extracted using a Zymogen RNA Isolation kit. DNA was removed using the RQ1 Promega RNAse free DNAse kit (M6101), and cDNA synthesized with the Life Tech High Capacity cDNA kit (#4368814). qPCR was performed with the SYBR-Green based detection system and sample amplification was done in triplicates on the BioRad-CFX. Results

We adapted Memon et al. 2018 protocol for the generation of human pluripotent stem cells into pancreatic progenitors with important modifications. Memon et al. 2018 protocol only describe how to reliably get to pancreatic progenitor stage from hPSCs. All the papers previously were geared towards deriving pancreatic endocrine cells (islets) from those pancreatic progenitor cells. Memon et al. does not make pancreatic exocrine cells from the pancreatic progenitors.

The new developments and invention described here show how to turn those pancreatic progenitor cells into pancreatic exocrine (Acinar and Ductal) cells by modulating different signaling pathways described I this invention and the timing of induction of ductal and acinar cell fates from pancreatic progenitor cells derived from pluripotent stem cells.

qPCR showed that several acinar markers (Mist1, Rbpj, Gata4, Ptf1a) were all expressed after four days of stage three media treatment, and pancreatic non-acinar markers (Ngn3, Pdx1, Sox9) downregulated after one day of stage 4 media treatment. From this it was decided that stage three would be 4 days.

Four days of stage three results in differentiation up to the pancreatic progenitor stage with decent efficiency. Quantification of ICC images or flow cytometry shows PDX1 expression in a percentage of cells and SOX9 a percentage of cells.

As a starting point a general pancreatic endoderm differentiation media was developed based on various previous protocols (Hohwieler et al., 2016; Takizawa-Shirasawa et al., 2013; Delaspre et al., 2013). There is disagreement between these prior protocols about the role of FGF and Notch signaling in acinar specific fate. We made an important decision to keep FGF10 and XXI as critical components of the acinar differentiation media, as we believed Notch signaling is important in the trunk versus tip fate assignment—though duct requires notch signaling.

We performed an experiment comparing the use of notch inhibitor XXI vs notch activator sDLL-1 with FGF10. Notch inhibition reduces PDX1 expression and increases islands of amylase positive cells. With the knowledge that FGF10 activates PDX1, we tested the importance of FGF10 concentration in concentrations of 0 ng/mL, 2.5 ng/mL, 20 ng/mL, and 50 ng/mL and found that the 20 ng showed the most CTRC and amylase positive cell expression. We added Wnt3a as it is important. We tested addition of noggin (BMP signaling agonist) and nicotinamide (amide form of Vitamin B3) and found that noggin nicotinamide addition decreases PDX1 expression and increases amylase and CTRC expression.

We developed a protocol for organoid generation by scraping and triturating to manually dissociate pancreatic progenitors at Day 9, and then seeding into 15 uL MATRIGEL bubbles plated into 96 well round bottom plates. The cells were fed with acinar media.

Organoids show lumen formation within one week after seeding into MATRIGEL bubbles. We have grown by day 57 and are starting to fuse. ICCs show that organoids express both acinar and ductal markers and do not express endocrine pancreatic markers. Organoids additionally show adhesion and polarization

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. Although the open-ended term “comprising,” as a synonym of terms such as including, containing, or having, is used herein to describe and claim the invention, the present invention, or embodiments thereof, may alternatively be described using alternative terms such as “consisting of”or “consisting essentially of.”

1. A method of differentiating induced pluripotent stem cells (iPSCs) into iPSC-derived pancreatic progenitor cells, comprising:
seeding induced pluripotent stem cells (iPSCs) on a solid medium coated with solubilized basement membrane preparation in the presence of serum-free, stabilized cell culture medium, and optionally a ROCK inhibitor;
culturing the cells in a first culture medium comprising a first base medium, Activin A, CHIR99021, and a ROCK Inhibitor;
culturing the cells in a second culture medium comprising the first base medium and Activin A, and bFGF (FGF-2);
culturing the cells in a third culture medium comprising the first base medium and FGF10, NOGGIN, and CHIRR99021;
culturing the cells in a fourth culture medium comprising a second base medium and FGF10, NOGGIN, All-trans Retinoic Acid, and SANT1,
wherein iPSC-derived pancreatic progenitor cells are produced.

2. The method of claim 1,
wherein Activin A, CHIR99021, and the ROCK Inhibitor in the first culture medium is at a concentration of about 100 ng/ml Activin A, about 2 uM CHIR99021 and about 10 uM of the ROCK inhibitor, or
wherein Activin A, and bFGF (FGF-2) in the second culture medium is at a concentration of about 100 ng/ml Activin A and about 5 ng/ml bFGF (FGF-2), or wherein FGF10, NOGGIN, and CHIRR99021 in the third culture medium is at a concentration of about 50 ng/ml FGF10, about 50 ng/ml NOGGIN and about 0.25 uM CHIRR99021, or
wherein FGF10, NOGGIN, All-trans Retinoic Acid, and SANT1 in the fourth culture medium is at a concentration of about 50 ng/ml FGF10, about 50 ng/ml NOGGIN, about 2 uM All-trans Retinoic Acid, and about 0.25 uM SANT1, or
any combination thereof.

3. (canceled)

4. (canceled)

5. (canceled)

6. The method of claim 1,
wherein the cells are cultured in the first culture medium for about 1 day, or
wherein the cells are cultured in the second culture medium for about 1 to 2 days, or
wherein the cells are cultured in the third culture medium for about 1 to 2 days, or
wherein the cells are cultured in the fourth culture medium for about 1 to 2 days, or
any combination of the above.

7. (canceled)

8. (canceled)

9. (canceled)

10. A method of differentiating induced pluripotent stem cells (iPSCs) into induced pancreatic (iPan) ductal cells, comprising:
performing the method of claim 1 or providing iPSC-derived pancreatic progenitor cells;
seeding the iPSC-derived pancreatic progenitor cells on a solid medium coated with solubilized basement membrane preparation in the presence of a fifth culture medium comprising a second base medium and a ROCK inhibitor; and
culturing the cells in a sixth culture medium comprising the second base medium and FGF10, EGF, and sDLL-1,
wherein iPan ductal cells are produced.

11. The method of claim 10, further comprising dissociating the iPSC-derived pancreatic progenitor cells before seeding the iPSC-derived pancreatic progenitor cells on the solid medium.

12. The method of claim 10,
wherein the ROCK inhibitor in the fifth culture medium is at a concentration of about 10 uM or
wherein FGF10, EGF, and sDLL-1 in the sixth culture medium is at a concentration of about 25 ng/ml FGF10, about 50 ng/ml EGF, and about 50 ng/ml sDLL-1.

13. (canceled)

14. The method of claim 10,
wherein the cells are cultured in the fifth culture medium for about 1 day, or
wherein the cells are cultured in the sixth culture medium for about 16 days, or both.

15. (canceled)

16. A method of differentiating induced pluripotent stem cells (iPSCs) into induced pancreatic endocrine cells, comprising:
performing the method of claim 1;
continuing to culturing the cells in the fourth culture medium comprising a second base medium and FGF10, NOGGIN, All-trans Retinoic Acid, and SANT1, for at least 2 additional days;
seeding the iPSC-derived pancreatic progenitor cells on a solid medium coated with solubilized basement membrane preparation in the presence of a fifth culture medium comprising a second base medium and a ROCK inhibitor;
culturing the cells in a sixth culture medium comprising the second base medium and noggin, EGF, and nicotinamide,
OR
providing iPSC-derived pancreatic progenitor cells that have been cultured in the fourth culture medium comprising a second base medium and FGF10, NOGGIN, All-trans Retinoic Acid, and SANT1, for at least 2 additional days;
seeding the iPSC-derived pancreatic progenitor cells on a solid medium coated with solubilized basement membrane preparation in the presence of a fifth culture medium comprising a second base medium and a ROCK inhibitor;
culturing the cells in a sixth culture medium comprising the second base medium and noggin, EGF, and nicotinamide,
wherein pancreatic endocrine cells are produced.

17. A method of producing pancreatic organoids comprising pancreatic ductal cells, comprising:
performing the method of claim 1 and dissociated the iPSC-derived pancreatic progenitor cells, or providing dissociated iPSC-derived pancreatic progenitor cells;
seeding the cells into a solubilized basement membrane preparation and plating the seeded cells onto a solid medium; and
culturing the cells in a sixth culture medium comprising the second base medium and FGF10, EGF, and sDLL-1.

18. The method of claim 17, wherein the iPSC-derived pancreatic progenitor cells are resuspended in the solubilized basement membrane preparation at a density of about 2 million cells/ML.

19. The method of claim 17,
wherein plating the seeded cells onto a solid medium comprises plating about 104, of cell suspension onto each solid medium, or
wherein the cells are cultured in the sixth culture medium for at least 2 weeks and given the sixth culture medium about every 2-3 days, or
wherein the solid medium is a U-bottom multi-well plate, or
any combination thereof.

20. (canceled)

21. (canceled)

22. A method of differentiating induced pluripotent stem cells (iPSCs) into iPSC derived pancreatic acinar cells, comprising:
performing the method of claim 1, or providing iPSC-derived pancreatic progenitor cells
seeding the iPSC-derived pancreatic progenitor cells into a solubilized basement membrane preparation and plating the seeded cells onto a solid medium;
culturing the cells in a seventh culture medium comprising a second base medium and XXI, FGF10, Noggin, Nicotinamide, and Murine Wnt3a,
wherein iPSC derived pancreatic acinar cells are produced.

23. The method of claim 22, wherein XXI, FGF10, Noggin, Nicotinamide, and Murine Wnt3a in the seventh culture medium is at a concentration of about 20 ng/mL FGF10, about 25 ng/mL Wnt3a, about 1 uM XXI, about 50 ng/mL Noggin, about 10 mM Nicotinamide.

24. A method of producing organoids comprising pancreatic acinar cells, comprising:
performing the method of claim 22 and dissociated the iPSC-derived pancreatic acinar cells, or providing dissociated iPSC-derived pancreatic acinar cells;
seeding the iPSC-derived pancreatic acinar cells into a solubilized basement membrane preparation and plating the seeded cells onto a solid medium;
culturing the cells in an eighth culture medium comprising a second base medium and FGF10, Noggin, Nicotinamide, and Murine Wnt3a.

25. The method of claim 24,
wherein seeding the iPSC-derived pancreatic acinar cells comprises seeding at a concentration of about 1-3×10\hat{ }6 cells per about 1040 μL of the solubilized basement membrane preparation, or
wherein plating the seeded cells onto a solid medium comprises plating in about 15 μL bubbles and allowing the plated cells to sit for about 15 minutes prior to culturing the cells in the eighth culture medium, or
wherein the cells are cultured in the eighth culture medium for about 44 days, and given the eighth culture medium about every other day, or
wherein the solid medium is a round-bottom multi-well plate, or
any combination thereof.

26. (canceled)

27. (canceled)

28. (canceled)

29. The method of claim 22, further comprising inducing PTF1A expression in the iPSC-derived pancreatic progenitor cells.

30. The method of claim 29, wherein inducing PTF1A expression in the iPSC-derived pancreatic progenitor cells comprises transducing the iPSC, pancreatic progenitor cells, or both with a lentivirus vector comprising a nucleic acid encoding PTF1A.

31. A method of claim 1,
wherein the solubilized basement membrane preparation is extracted from the Engelbreth-Holm-Swarm (EHS) mouse sarcoma, or is MATRIGEL matrix, or
wherein the ROCK inhibitor is Y27632 ROCK Inhibitor, or
wherein serum-free, stabilized cell culture medium is mTeSR plus medium, or any combination thereof.

32. (canceled)

33. (canceled)

34. (canceled)

35. A method of claim 1,
wherein the first base medium comprises an agent from the group consisting of MCDB 131, Glutamax, Vitamin C, BSA, sodium bicarbonate, antibiotic, and combinations thereof,
wherein the second base medium comprises an agent from the group consisting of DMEM, antibiotic, B27 without vitamin A, vitamin C and combination thereof.

36. A method of claim 1,
wherein the first base medium comprises MCDB 131, Glutamax, Vitamin C, BSA, sodium bicarbonate, and antibiotic, or
wherein the second base medium comprises DMEM, antibiotic, B27 without vitamin A, and vitamin C, or both.

37. The method of claim 35, wherein the Glutamax, Vitamin C, BSA, sodium bicarbonate, and antibiotic are at a concentration of about 1×Glutamax, about 1×Antibiotic, about 0.5% Bovine Serum Albumin, about 1.5 mg/ml Sodium Bicarbonate and about 250 uM Vitamin C.

38. (canceled)

39. A method of claim 35, wherein the second base medium comprises DMEM, antibiotic, B27 without vitamin A, and vitamin C.

40. The method of claim 35, wherein the antibiotic, B27 without vitamin A, and uM vitamin C are at a concentration of about 1×antibiotic, about 1×B27 without vitamin A, and about 250 uM vitamin C.

41. A method of claim 1, wherein the antibiotic is a Pen-Strep Antibiotic.

42. An induced pluripotent stem cell (iPSC) derived pancreatic progenitor cell made by a method of claim 1.

43. An induced pluripotent stem cell (iPSC) derived iPan ductal cell made by a method of claim 10.

44. An induced pluripotent stem cell (iPSC) derived organoid comprising pancreatic ductal cells made by a method of claim 17.

45. An induced pluripotent stem cell (iPSC) derived pancreatic acinar cell made by a method of claim 22.

46. An induced pluripotent stem cell (iPSC) derived organoid comprising pancreatic acinar cells made by a method of claim 24.

47. A model, comprising:
a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; and
a fluidic device, or a cell culture plate, or a multi-well culture plate,
wherein the test agent and the population of cells are in contact in a fluidic device the cell culture plate, or the multi-well culture plate.

48. The model of claim 47,
wherein the fluidic device, the cell culture plate, or the multi-well culture plate is a Transwell system, or
wherein the fluidic device is a microfluidic device, or
wherein the microfluidic device is an organ chip.

49. (canceled)

50. (canceled)

51. A device, comprising:
a membrane comprising a top surface and a bottom surface;
a first channel in fluidic communication with the top surface of the membrane;
a second channel in fluidic communication with the bottom surface of the membrane, wherein the first and second channels each comprises a surface that is parallel to the membrane;
a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells in the first channel or the second channel; and
optionally, a second population of cells in the first channel or the second channel,
wherein the first population of cells and the second population of cells are in different channels, and
wherein the first population of cells and the second population of cells are not the same type of cells,
optionally, wherein the second population of cells are selected from the group consisting of endocrine cells, stellate cells endothelial cells, pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells.

52. (canceled)

53. A device of claim 51, further comprising:
at least one inlet port adapted for fluid entering the at least one inlet port; and
at least one outlet port adapted for fluid exiting the at least one outlet port.

54. A device, comprising:
a top chamber;
a bottom chamber;
a membrane between the top chamber and the bottom chamber;
a first channel fluidically coupled to the top chamber;
a second channel fluidically coupled to the bottom chamber;
a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells in the first channel or the second channel; and
optionally, a second population of cells in the first channel or the second channel,
wherein the first population of cells and second population of cells are in different channels, and
wherein the first population of cells and the second population of cells are not the same type of cells,
optionally, wherein the second population of cells are selected from the group consisting of endocrine cells, stellate cells endothelial cells, pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells.

55. (canceled)

56. A device of claim 51, wherein the first and second channels comprise polydimethylciloxane or wherein the first channel and the second channel are microfluidic channels.

57. (canceled)

58. An organ chip device, comprising:
a first population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells;
optionally, a second population of cells; and
a membrane separating the first population of cells and the second population of cells,
wherein the first population of cells and the second population of cells are not the same type of cells,
optionally, wherein the membrane comprises polydimethylciloxane.

59. (canceled)

60. A device of claim 51, further comprising one or more gels and the population of cells having been seeded on top of or into the one or more gels.

61. A device of claim 51, wherein the first population of cells, or the second population of cells or both are patient specific or wherein the first population of cells, or the second population of cells or both express a fluorescent reporter.

62. (canceled)

63. A method of assessing a test agent, comprising:
contacting the test agent to a device of claim 51, wherein the device comprises
a population of cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, and pancreatic acinar cells derived from pancreatic progenitor cells, and
optionally, a second population of cells;
measuring a parameter; and
assessing the test agent based on the measured parameter.

64. The method of claim 63,
wherein measuring the parameter comprises measuring a phenotype of interest, expression level of a gene of interest, or expression level of a protein of interest, or combinations thereof, or
wherein contacting the population of cells with the test agent comprises culturing the population of cells in the presence of culture media flowing through the device, or
wherein the first population of cells or the second population of cells, or both are patient specific and the method models patient-specific parameters.

65. (canceled)

66. (canceled)

67. A method of producing a device of claim 58, comprising:
seeding the first population of cells on one surface of the membrane in the device; and optionally, seeding second population of cells on the other surface of the membrane in the device;
OR
seeding the first population of cells in one chamber in the device; and optionally, seeding the second population of cells in the other chamber in the device.
